(19) United States
(12) Patent Application Publication
(10) Pub. No.: US 2011/0033931 A1
(43) Pub. Date: Feb. 10, 2011

(54) DE-DIFFERENTIATION OF HUMAN CELLS

(75) Inventors: Philip Schwartz, Orange, CA (US); Rampyari Walia, El Cajon, CA (US)

Correspondence Address: KNOBBE MARTENS OLSON & BEAR LLP, 2040 MAIN STREET, FOURTEENTH FLOOR, IRVINE, CA, 92614, US

(73) Assignee: CHILDREN'S HOSPITAL OF ORANGE COUNTY, ORANGE, CA (US)

(21) Appl. No.: 12/740,672

(22) PCT Filed: Nov. 26, 2008
(86) PCT No.: PCT/US08/84893

§ 371 Date: Oct. 25, 2010

Related U.S. Application Data
(60) Provisional application No. 60/991,197, filed on Nov. 29, 2007.

(51) Int. Cl. C12N 5/071

(52) U.S. Cl. 435/377

(57) ABSTRACT

Methods of de-differentiating somatic cells to an embryonic stem cell state comprising direct delivery of a protein into the somatic cell, wherein the protein effects de-differentiation of the somatic cell to an embryonic stem cell phenotype.

### RELATED APPLICATIONS

This application claims the benefit of U.S. Provisional Application No. 60/991,197 filed Nov. 29, 2007, which is hereby expressly incorporated by reference in its entirety.

### FIELD OF THE INVENTION

The invention relates to methods of de-differentiating somatic cells to an embryonic stem cell state.

### DESCRIPTION OF THE RELATED ART

Epigenetic reprogramming of somatic cells into embryonic stem (ES) cells has attracted much attention because of the potential for customized transplantation therapy, as cellular derivatives of reprogrammed cells will not be rejected by the donor (Hochedlinger, K. and Jaenisch R. 2003 N Eng L Med 349:275-286; Yang, X. et al. 2007 Nature Genet. 39:295-302). Thus far, somatic cell nuclear transfer and fusion of fibroblasts with ES cells have been shown to promote the epigenetic reprogramming of the donor genome to an embryonic state (Hochedlinger, K. and Jaenisch R. 2006 Nature 441:1061-1067; Tada M, et al. 2001 Curr Biol 11:1553-1558; Cowan, C. A. et al. 2005 Science 309:1369-1373). However, the therapeutic application of either approach has been hindered by technical complications as well as ethical objections (Jaenische, R 2004 N Engl J Med 351:2787-2791). Recently, a major breakthrough was reported whereby expression of the transcription factors Oct4, Sox2, c-Myc and Klf4 was shown to induce mouse and human fibroblasts to become pluripotent stem cells (designated as induced pluripotent stem (iPS) cells) (Takahashi, K and Yamanaka, S 2006 Cell 126:663-676; Takahashi, K et al. 2007 Cell 131:861-872). The iPS cells were isolated by selection for activation of Fbx15 (also called Fbxo15), which is a downstream gene of Oct4. DNA methylation, gene expression and chromatin state of such induced reprogrammed stem cells are similar to those of ES cells (Wernig, M. et al. 2007 Nature 448:318-324. Such cells, derived from mouse fibroblasts, can form viable chimaeras, contribute to the germ line and generate live late-term embryos when injected into tetraploid blastocysts. Moreover, the biological potency and epigenetic state of in-vitro-reprogrammed induced pluripotent stem cells are indistinguishable from those of ES cells.

The reprogramming mechanism is not restricted to fibroblast type cultures, as a variety of reports have shown the generation of iPSCs from adult mouse hepatocytes, gastric epithelial cells, pancreatic beta cells and terminally mature B lymphocytes. The biologic potential and epigenetic state of iPSCs are indistinguishable from those of human embryonic stem cell (hES) cultures. Indeed iPSCs express pluripotency markers, form teratomas and contribute to all germ layer cell types in chimeric animals.

The iPSC technology is expected to revolutionize modern medicine and clinical research. Patient-specific stem cells that can differentiate into virtually any tissue cell type in the body can now theoretically be created from the fibroblast cell of the donor. This obviates potential concern regarding immune rejection of transplanted stem cells by the patient, as the donor acts as the recipient for the iPSCs generated from their own fibroblasts. In addition, iPSC technology will allow major advances in the understanding and treatment of diseases for which we currently have limited insight. This is because iPSC technology will allow the establishment of a large number of disease specific cell lines for study from a skin biopsy or tissue repository. In addition, iPSC technology will help alleviate the public and political controversy and fear surrounding stem cell research as it represents an important means of deriving pluripotent stem (PS) cells independent of human embryos.

There are several obstacles in the current iPSC generation model that need to be overcome. These include the irreversible genetic modification (e.g., with lentiviral transgenes) of the fibroblast genome and subsequently the iPSC genome and low efficiency reprogramming/induction seen using the currently known reprogramming factors. Although lentiviral vectors are extremely useful research tools in that they can transduce dividing and non-dividing cells, there are major safety concerns regarding their clinical use. This is because lentiviral vectors integrate randomly into the genome and issues related to vector insertional mutagenesis, vector insertional dysregulation of cellular genes and vector mobilization arise.

### SEGUE TO THE INVENTION

Here, we describe the application of a preexisting novel nuclear targeting reagent to the generation of iPSCs. This novel system has been used previously in several published studies to deliver functional proteins into the nucleus of cells (Nandan, D. et al. 2002 J Biol Chem 277:50190-50197; Sendide, K. et al. 2005 J Immunol 175:5324-5332; Miao, E. A. et al. 2006 Nature Immunology 7:569-575; Tanaka H. et al. 2006 Stem Cells 24:2592-2602; Soualhine H. et al. 2007 J Immunol 179:5137-5145). This technology can be applied to iPSC generation by delivering known nuclear reprogramming factors (e.g., Oct3/4, Sox2, Nanog, c-Myc, etc) as recombinant proteins into the nuclei of human fibroblasts and selecting for the generation of iPSCs. Combinations of different transcription factor subsets, all centered around Oct3/4 and Sox2 expression, are sufficient to trigger the induction of reprogramming when delivered by lentiviral transduction. Functional delivery of the same combination(s) of reprogramming protein factors directly into the nucleus of fibroblasts with this reagent is envisioned as triggering the onset of reprogramming. The generation of iPSCs, independent of lentiviral and integrative delivery mechanisms, alleviates safety concerns regarding the use of genetically modified iPSCs for transplantation and facilitates a more rapid movement of patient-specific iPSCs into the clinic.

### SUMMARY OF THE INVENTION

The present invention relates to a method of de-differentiating somatic cells to an embryonic stem cell state comprising direct delivery of a protein into the somatic cell, wherein the protein effects de-differentiation of the somatic cell to an embryonic stem cell phenotype.

In some embodiments, the method of de-differentiating somatic cells to an embryonic stem cell state comprises direct delivery of a protein selected from the group consisting of Oct3/4, Sox2, Nanog, Stat3, E-Ras, c-Myc, Klf4, β-catenin and Lin28 into the somatic cell.

In some embodiments, the method of de-differentiating somatic cells to an embryonic stem cell state comprises direct delivery of a mutant, variant or a derivative of a protein or polypeptide that is able to induce and maintain the embryonic stem cell phenotype.

In some embodiments, the method of de-differentiating somatic cells to an embryonic stem cell state comprises direct delivery of a protein to the cells with a Profect protein delivery reagent selected from the group consisting of Profect-P1 and Profect-P2.

In some embodiments, the proteins or polypeptides used to induce and maintain the ES cell phenotype are identified by differential gene expression analysis.

### BRIEF DESCRIPTION OF THE DRAWINGS

FIG. 1. Schematic illustration of Oct3/4 Protein.

FIG. 2. Schematic illustration of Sox2 Protein.

FIG. 3. Schematic illustration of Nanog Protein.

FIG. 4. Schematic illustration of Stat3 Protein.

FIG. 5. Amino acid sequence alignment of human ERas (hERas, SEQ ID NO: 8); mouse ERas (mERas, SEQ ID NO: 13); and Harvey rat sarcoma virus oncogene (hRas, SEQ ID NO: 14).

FIG. 6. Schematic illustration of c-Myc Protein.

FIG. 7. Schematic illustration of Klf4 Protein.

FIG. 8. Schematic illustration of β-catenin Protein.

FIG. 9. Schematic illustration of Lin28 Protein.

FIG. 10. Diagram of Profect-mediated protein delivery.

FIG. 11. Nuclear Delivery of Full-Length Nanog into human fibroblasts using Profect P2 Reagent.

FIG. 12. Induced expression of full length human Oct-4 protein as a GST fusion protein using IPTG and the pET expression system.

FIG. 13. Differentiation of human pluripotent stem cells into neural stem cells (NSC). Left panel: Neurally-induced embryoid bodies derived from ePSCs and plated onto Matrigel show classic neural rosette formation. Center panel: Same field as left panel but a higher magnification. Right panel: neural rosettes stain positively for the NSC markers Sox1 and N-cadherin.

FIG. 14. Amino Acid Sequence Alignment of two human Oct3/4 isoforms, Accession Nos. NP_{—}002692 (SEQ ID NO: 1) and NP_{—}976034 (SEQ ID NO: 2). Consensus symbols: “*”, residues in column are identical in all sequences; “:”, conserved substitutions; “.”, semi-conserved substitutions.

FIG. 15. Amino acid sequence of human Sox2, Accession No. NP_{—}003097 (SEQ ID NO: 3).

FIG. 16. Amino acid sequence of human Nanog, Accession No. NP_{—}079141 (SEQ ID NO: 4).

FIG. 17. Amino Acid Sequence Alignment of three human Stat3 isoforms, Accession Nos. NP_{—}644805 (SEQ ID NO: 5), NP_{—}003141 (SEQ ID NO: 6) and NP_{—}998827 (SEQ ID NO: 7). Consensus symbols: “*”, residues in column are identical in all sequences; “:”, conserved substitutions; “.”, semi-conserved substitutions.

FIG. 18. Amino acid sequence of human E-Ras, Accession No. NP_{—}853510 (SEQ ID NO: 8).

FIG. 19. Amino acid sequence of human c-Myc, Accession No. 0907235A (SEQ ID NO: 9).

FIG. 20. Amino acid sequence of human Klf4, Accession No. NP_{—}004226 (SEQ ID NO: 10).

FIG. 21. Amino acid sequence of human β-catenin, Accession No. NP_{—}001895 (SEQ ID NO: 11).

FIG. 22. Amino acid sequence of human Lin28, Accession No. NP_{—}078950 (SEQ ID NO: 12).

### DETAILED DESCRIPTION OF THE PREFERRED EMBODIMENT

The basis of this invention is the intracellular insertion of specific proteins into the nucleus of cells to cause the cells to de-differentiate. Recent data has shown that, in mouse cells, forced expression of certain repressed genes causes fully differentiated mouse cells to de-differentiate to an embryonic stem cell phenotype. Two very important aspects of this data warrant observation. First, the expression of these genes is transient, suggesting that the proteins that they encode need only be present for a limited period of time to effect the de-differentiation. Second, since the method used a viral-based, genetic manipulation, it potentially provides cells of high research interest, but is unlikely to be useful for generating cells of therapeutic interest.

We disclose here a method for directly inserting the proteins of interest into cells to effect de-differentiation of the cells to an embryonic state. These proteins of interest may or may not be identical to those described in the mouse and human studies. Gene microarray data may be used to identify other proteins of interest.

### DEFINITIONS

Unless defined otherwise, technical and scientific terms used herein have the same meaning as commonly understood by one of ordinary skill in the art to which this invention belongs. See, e.g., Singleton P and Sainsbury D., Dictionary of Microbiology and Molecular Biology 3rd ed., J. Wiley & Sons, Chichester, N.Y., 2001.

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

### Known De-Differentiation Genes

Several transcription factors, including Oct3/4 (Nichols, J. et al. 1998 Cell 95:379-391; Niwa, H. et al., 2000 Nat Genet. 24:372-376), and Sox2 (Avilion, A. A. et al., 2003 Genes Dev 17:126-140) and Nanog (Chambers, I. et al. 2003 Cell 113:643-655 and Mitsui, K. et al. 2003 Cell 113:631-642) function in the maintenance of pluripotency in both early embryos and ES cells. Several genes that are frequently upregulated in tumors, such as Stat3 (Matsuda, T. et al. 1999 EMBO J. 18:4261-4269 and Niwa, H. et al. 1998 Genes Dev 12:2048-2060), E-Ras (Takahashi, K. et al. 2003 Nature 423:541-545), c-myc (Cartwright, P. et al. 2005 Development 132:885-896), Klf4 (Li, Y. et al. 2005 Blood 105:635-637) and β-catenin (Kielman, M. F. et al. 2002 Nat Genet. 32:594-605 and Sato, N. et al. 2004 Nat Med 10:55-63) have been shown to contribute to the long-term maintenance of the ES cell phenotype and the rapid proliferation of ES cells in culture.

### Oct3/4

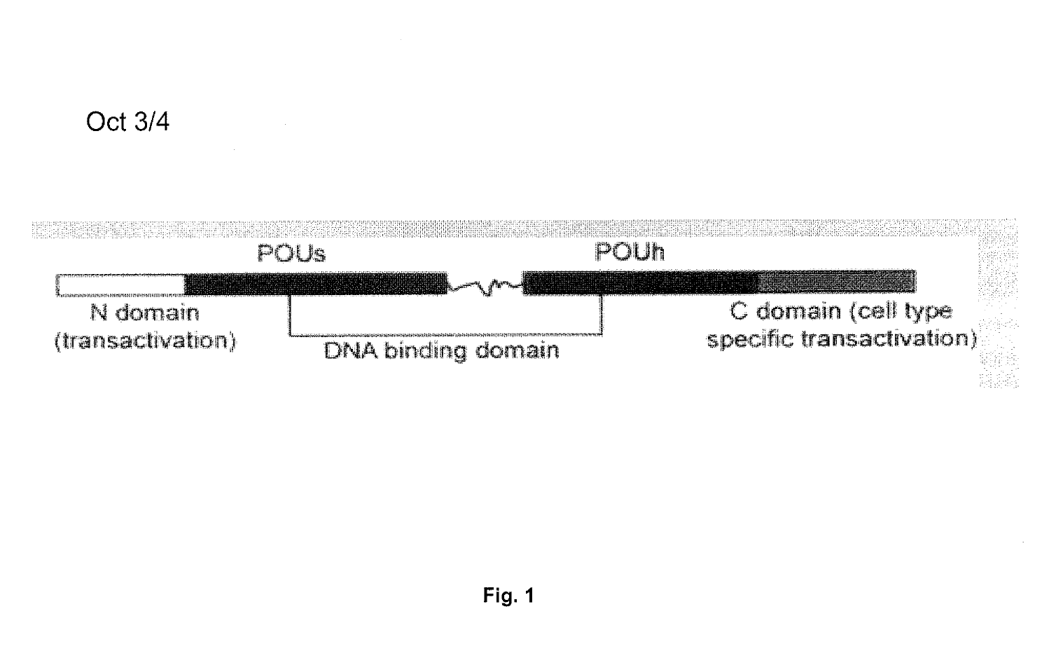

The (Pit1-Oct1/2-Unc86) POU factor Oct4 (also known as Oct3) is distinguished by exclusive expression in blastomeres, pluripotent early embryo cells, and the germ cell lineage (Pan, G. J. et al. 2002 Cell Research 12:321-329). The hallmark feature of the POU family of transcription factors is the POU domain, which consists of two structurally independent subdomains: a 75 amino acid amino-terminal POU specific (POUs) region and a 60 amino-acid carboxyl-terminal homeodomain (POUh) (see FIG. 1). Both domains make specific contact with DNA through a helix-turn-helix structure and are connected by a variable linker of 15 to 56 amino-acids. Regions outside the POU domain are not critical for DNA binding and exhibit little sequence conservation. The N-terminal domain (N domain) is rich in proline and acidic residues, while the C-terminal domain (C domain) is rich in proline, serine and threonine residues. The N domain has traditionally been accepted for its role in transactivation. More recent data suggest that the C domain also plays a role in transactivation. Investigators replaced the POU DNA binding domain with those from other transcription factors, for example, the heterologous yeast Gal4 DNA binding domain. This replacement does not affect its transactivation function, suggesting that general transactivation function can be transferred to unrelated DNA binding domains. The activity of Oct4 C domain is cell type specific and is regulated through phosphorylation, whereas the N domain is not. The cell type specificity is observed only if the C domain is linked to the POU domains of Oct-4 and Oct-2, but not to Pit-1 or the Gal4 DNA binding domain. This finding suggests that Oct4 POU-domain may function differently by serving as interaction sites for cell type-specific regulatory factors.

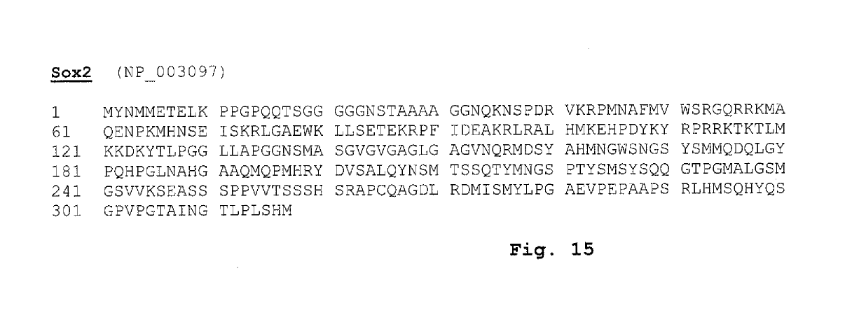

Since the cell-type-specific activity of regulatory factors ensures the expression of target genes in an orderly fashion during development, Oct4 and its functional partners may be regulated in a specific manner throughout mammalian embryogenesis. Indeed, Oct4 is expressed by germ cells from the totipotent zygote to the highly specialized oocyte. It is likely that Oct4 may function in concert with other regulators to activate specific target genes in specific cell types at defined developmental stages. The fact that the N domain differs from the C domain in activity and cell type specificity may help explain the functional diversity for Oct4. Furthermore, the C domain may activate certain targets, which do not respond to the N domain during development. FIG. 14 shows an amino acid sequence alignment of two isoforms of human Oct 3/4.

### Sox2

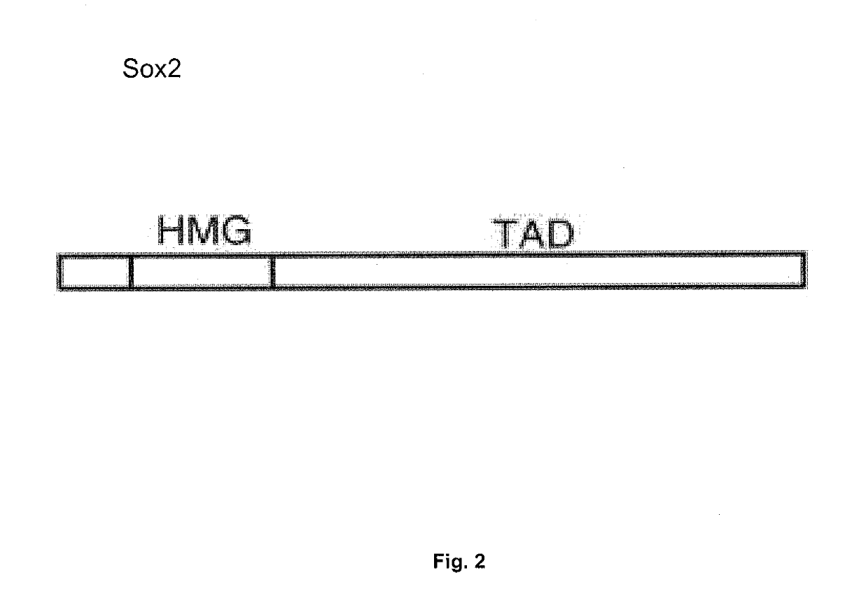

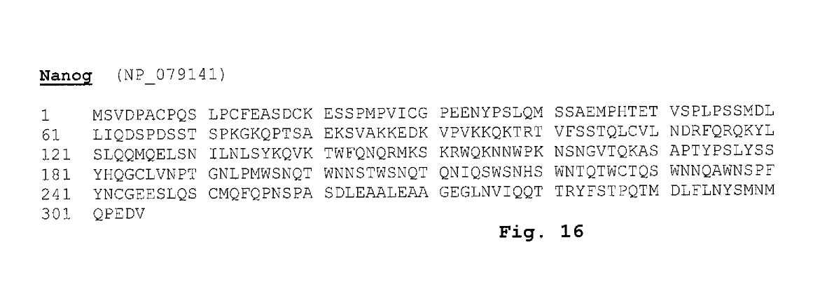

The transcription factor Sox2 has been implicated in the regulation of Fgf4 expression (Avilion A. A. et al. 2003 Genes & Development 17:126-140). Sox2 is a member of the Sox (SRY-related HMG box) gene family that encodes transcription factors with a single HMG DNA-binding domain. SOX2 belongs to the SOX B1 subgroup, which also includes SOX1 and SOX3, based on homology within and outside the HMG box. Several lines of evidence indicate that SOX2 may act to maintain or preserve developmental potential. For example, Sox2 expression is associated with uncommitted dividing stem and precursor cells of the developing central nervous system (CNS) and indeed can be used to isolate such cells. Sox2 also marks the pluripotent lineage of the early mouse embryo. Referring to FIG. 2, the Sox2 protein contains an HMG DNA binding domain and a transactivating domain (TAD). FIG. 15 shows the amino acid sequence of human Sox2.

### Nanog

NANOG regulates pluripotency mainly as a transcription repressor for downstream genes (Pan, G. and Pei, B. 2005 J Biol Chem 280:1401-1407). NANOG appears to function in parallel with STAT3 and be sufficient for maintaining stem cell pluripotency. NANOG not only inhibits the differentiation of stem cells into endoderm but also actively maintains pluripotency, in contrast to the role of Oct4 as a blacker of differentiation of inner cell mass and ES cells into trophectoderm. NANOG has been proposed as a determinant of pluripotency for inner cell mass and ES cells. Because differentiation and self-renewal are likely to be regulated through the expression of mutually exclusive genes, NANOG may assume a bifunctional role to repress those genes important for differentiation and activate the ones necessary for self-renewal.

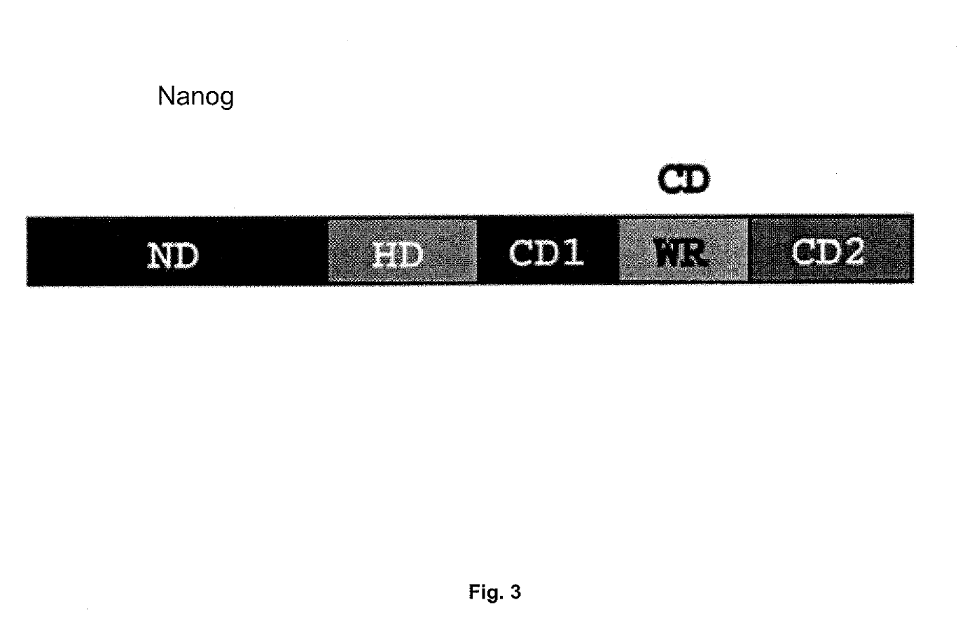

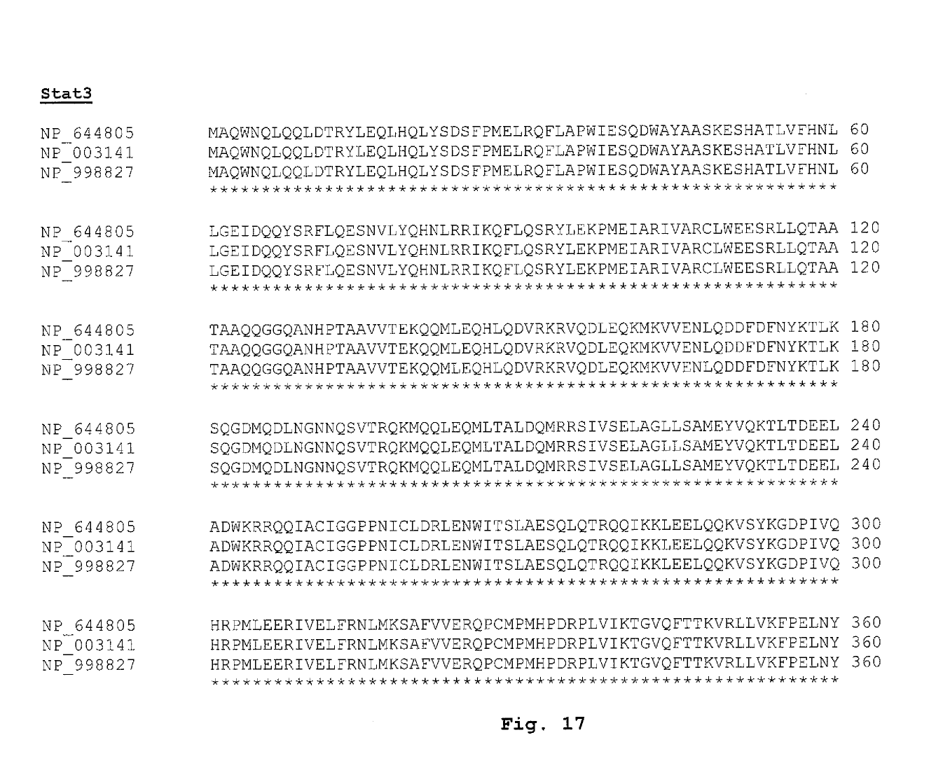

Referring to FIG. 3, NANOG is a multidomain protein with a well conserved Nk-2 homeodomain, an N-terminal transactivation domain (ND) and a C-terminal transactivation domain (CD). A signature 60-residue homeodomain is proposed to bind DNA and interact with other proteins as demonstrated for Oct4. The N-terminal domain contains 95 residues rich in Ser and Thr and acidic residues found in typical transactivators. NANOG has two unusually strong activation domains embedded in its C terminus. These two transactivators are named WR and CD2. Whereas CD2 contains no obvious structural motif, the WR or Trp repeat contains 10 pentapeptide repeats starting with a Trp in each unit. Substitution of Trp with Ala in each repeat completely abolished its activity, whereas mutations at the conserved Ser, Gln, and Asn has relatively minor or no effect on WR activity. Data suggest that either WR or CD2 is sufficient for NANOG to function as a transactivator. FIG. 16 shows the amino acid sequence of human Nanog.

### Stat3

STATs are a family of latent cytoplasmic transcription factors that were named by virtue of their novel and unique dual functions as signaling molecules in the cytoplasm and as transcription factors after nuclear translocation (Ma, J. et al. 2003 J Biol Chem 278:29252-29260). Stat proteins are primarily located in the cytoplasm. Upon cytokine stimulation, Stat proteins are recruited to the cytokine receptors and phosphorylated by the receptor-associated tyrosine kinases, Janus kinases, on a single tyrosine residue at the C termini. Stat proteins form homo- or heterodimers via reciprocal interactions between the SH2 domains and the phosphotyrosine and translocate into the nucleus, where they bind to DNA and regulate transcription of their target genes.

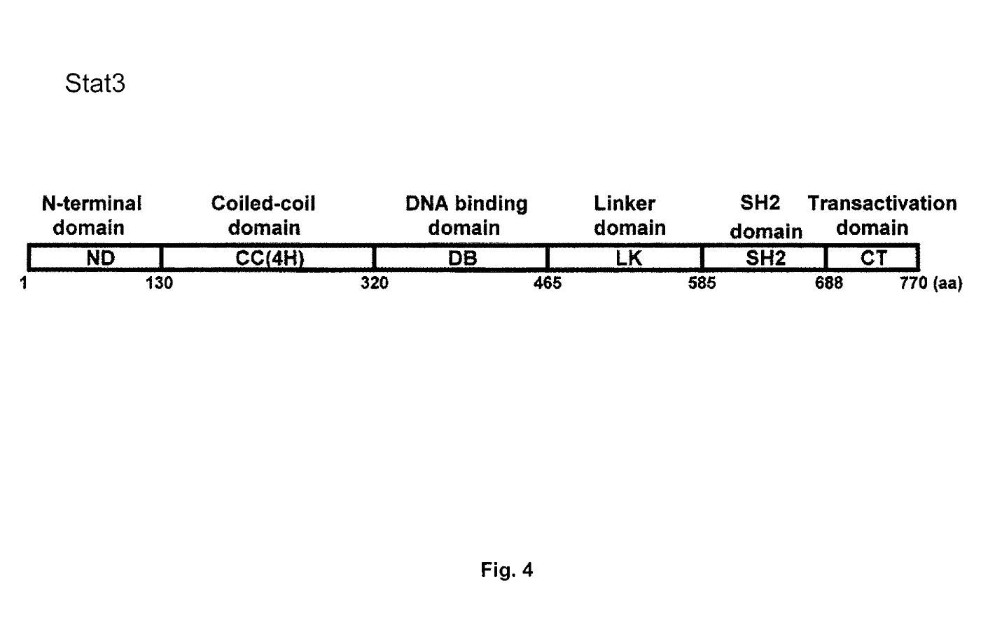

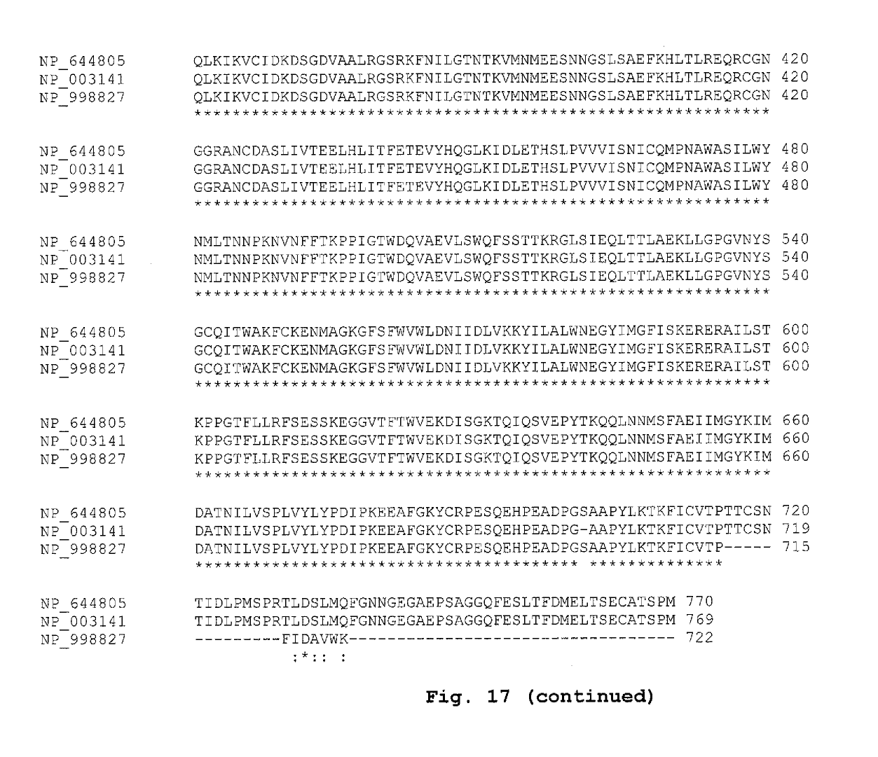

Seven known mammalian Stat proteins, denoted by Stat1, Stat2, Stat3, Stat4, Stat5a, Stat5b, and Stat6, have been identified. They are activated by various cytokines and growth factors and play important roles in diverse cellular processes such as the antiviral protection, immune responses, cell growth, and apoptosis by regulating expression of numerous genes. As shown in FIG. 4, Stat3 has several conserved functional domains including an N-terminal domain (ND), a coiled-coil domain (CC), a DNA binding domain (DB), and a linker domain (LK), followed by an SH2 domain and a C-terminal transactivation domain (CT). Stat3 plays a broad role in a variety of biological responses such as cell growth, transformation, survival, and early embryonic development. FIG. 17 shows an amino acid sequence alignment of three isoforms of human Stat3.

### E-Ras

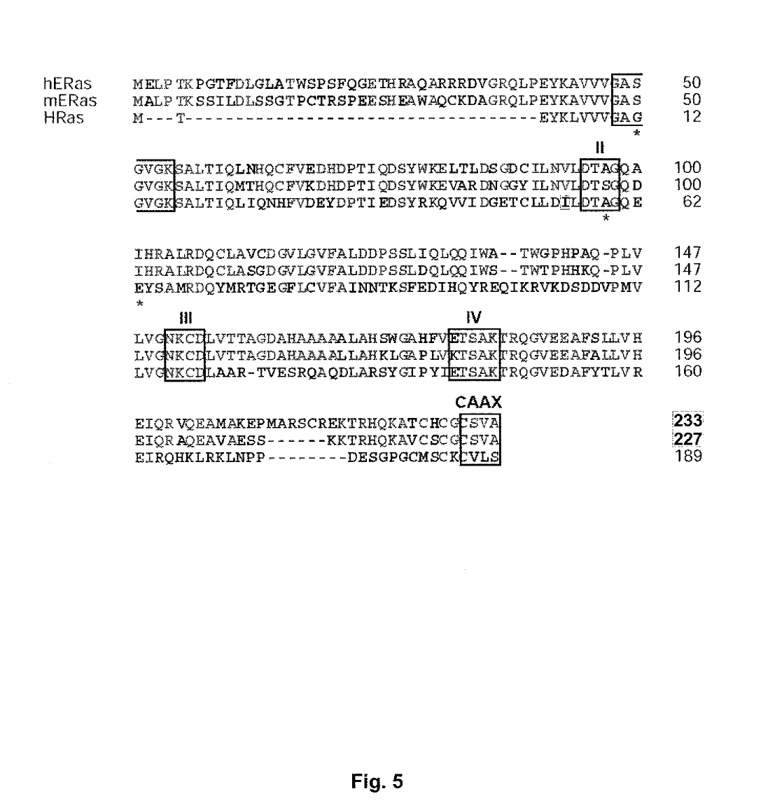

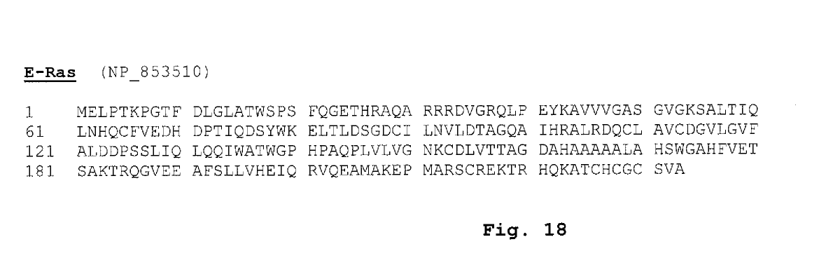

Mouse ES cells specifically express a Ras-like gene named Eras (Takahashi, K. et al. 2003 Nature 423:541-545). Human HRasp, a recognized pseudogene, encodes the human ortholog of ERas. This protein contains amino-acid residues identical to those present in active mutants of Ras and causes oncogenic transformation in NIH 3T3 cells. ERas interacts with phosphatidylinositol-3-OH kinase but not with Raf. ERas-null ES cells maintain pluripotency but show significantly reduced growth and tumorigenicity, which are rescued by expression of ERas complementary DNA or by activated phosphatidylinositol-3-OH kinase. The transforming oncogene ERas is important in the tumor-like growth properties of ES cells. Murine ERas is a protein of 227 amino acids with 43%, 46% and 47% identity to HRas, KRas and NRas, respectively. Five domains essential for small G proteins are highly conserved in the three proteins, including a CAAX motif (FIG. 5). FIG. 18 shows the amino acid sequence of human E-Ras.

c-Myc

A potential role for Myc in ES cell maintenance is suggested by two reports. First, expression of an RLF/L-myc minigene that frequently arises from a chromosomal translocation event in human small lung carcinomas, delays ES cell differentiation and interferes with early embryonic development (MacLean-Hunter et al. 1994 Oncogene 9:3509-3517). Second, elevated Myc activity is able to block the differentiation of multiple cell lineages (Selvakumaran et al. 1996 Blood 4:1248-1256).

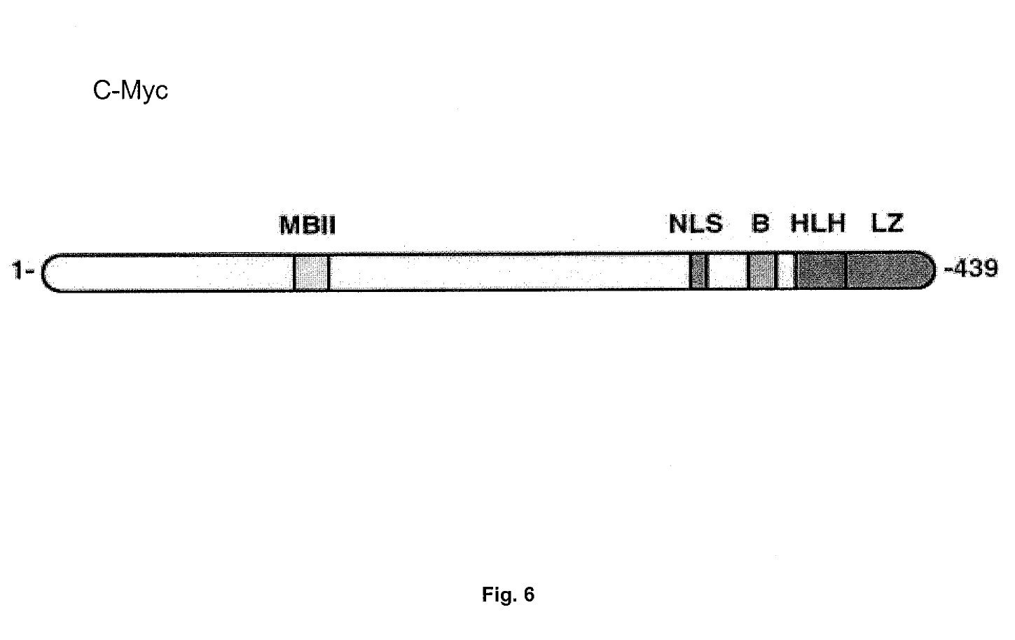

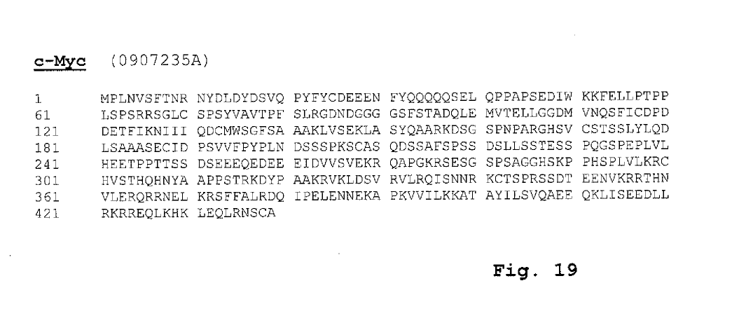

Referring to FIG. 6, functional domains of c-MYC protein include MBII, the highly conserved MYC homology box II region; a nuclear localization signal (NLS); B, the basic DNA binding motif; HLH, the helix-loop-helix domain essential for dimerization with MAX and a leucine zipper motif (LZ). FIG. 19 shows the amino acid sequence of human c-Myc.

### Klf4

Human Krüppel-like factor (KLF4) (formerly known as gut-enriched KLF or epithelial zinc finger, EZF) was first identified from human umbilical vein endothelial cell cDNA library by using a DNA probe containing the zinc finger region of human erythroid Krüppel-like factor (EKLF, KLF 1) (Wei, D et al. 2006 Carcinogenesis 27:23-31). The cDNA of KLF4 encodes a protein containing 470 amino acids with a predicted molecular mass of 50 kDa. Several functional domains have been characterized in the KLF4 protein, including an acidic transcriptional activation domain at the N-terminus; the carboxyl DNA-binding domain, which consists of 81 highly conserved amino acids that form three C2H2 zinc fingers that exhibit homology with the D. melanogaster segmentation gene product Krüppel; and nuclear localization signal and transcriptional repression domains at the N-terminus next to the three zinc fingers. In addition, there is a potential PEST sequence located between the transcriptional activation and transcriptional inhibitory domains, indicating that KLF4 may be degraded through ubiquitin-proteosome pathway.

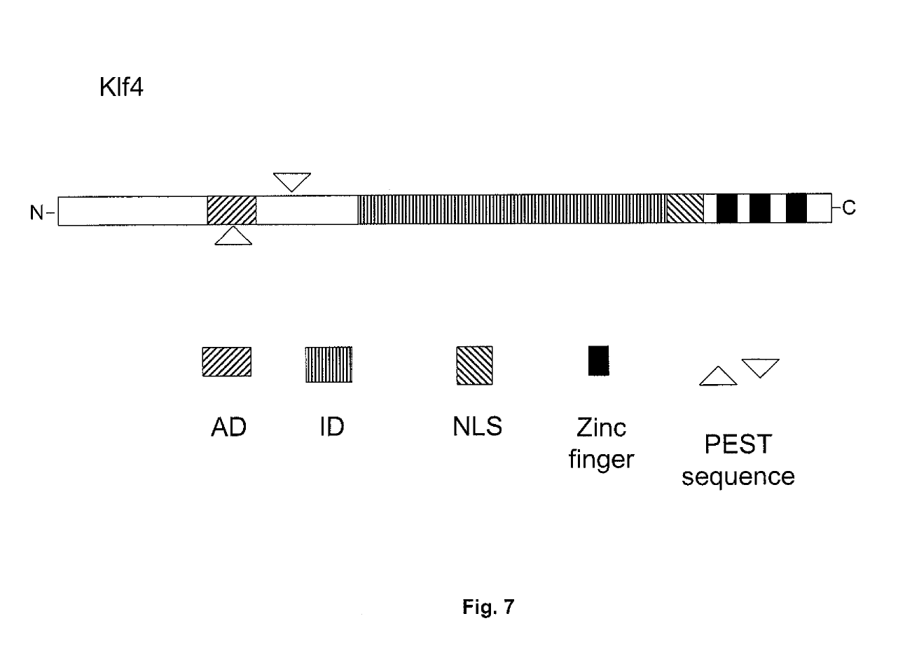

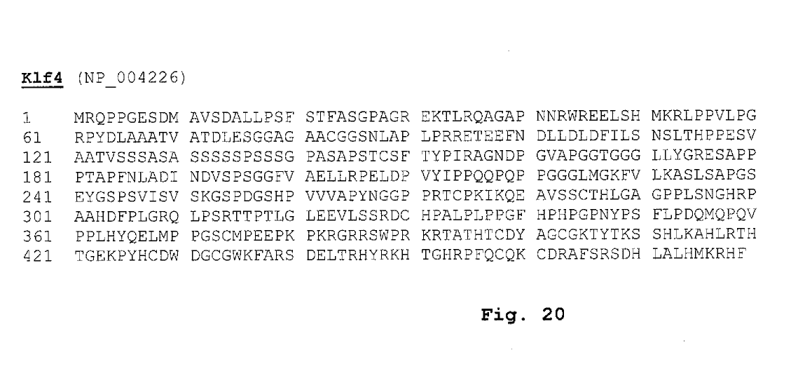

Referring to FIG. 7, the Klf4 open reading frame encodes a protein of about 470 amino acids with several functional domains, including the transcriptional activation domain (AD), transcriptional inhibitory domain (ID), zinc finger DNA-binding domain, nuclear localization signal (NLS) and potential PEST sequence. FIG. 20 shows the amino acid sequence of human Klf4.

### β-Catenin

β-catenin is a multifunctional adaptor protein involved in cadherin-mediated cell-cell adhesion and in responding to the activation of several signal transduction pathways, including Wnts, Akt/protein kinase B, epidermal growth factor (EGF), insulin-like growth factor, integrin-linked kinase, nuclear factor-κB, p53, Pin1, PTEN, FP(B) prostanoid receptor, nuclear hormone receptors such as peroxisome proliferator-activated receptors (PPARs), androgen receptor (AR) and retinoic acid receptor (RAR), and oxidative stress. Its role is best characterized in the canonical Wnt signaling pathway. β-catenin signaling has been implicated in the maintenance and self-renewal of stem or progenitor cells in various tissues including skin, blood, and gut.

The Wnt signal-transduction pathway induces the nuclear translocation of membrane-bound β-catenin and has a key role in cell-fate determination (Kielman, M. F. et al. 2002 Nature Genetics 32:594-605). Tight somatic regulation of this signal is essential, as uncontrolled nuclear accumulation of β-catenin can cause developmental defects and tumorigenesis in the adult organism. The adenomatous polyposis coli gene (APC) is a major controller of the Wnt pathway and is essential to prevent tumorigenesis in a variety of tissues and organs. The ability and sensitivity of ES cells to differentiate into the three germ layers is inhibited by increased doses of β-catenin by specific Apc mutations. These range from a severe differentiation blockade in Apc alleles completely deficient in β-catenin regulation to more specific neuroectodermal, dorsal mesodermal and endodermal defects in more hypomorphic alleles. Evidence suggests that constitutive activation of the Apc/β-catenin signaling pathway may result in differentiation defects in tissue homeostasis, and possibly underlies tumorigenesis in the colon and other self-renewing tissues. Thus, different doses of β-catenin correlate with differentiation potential.

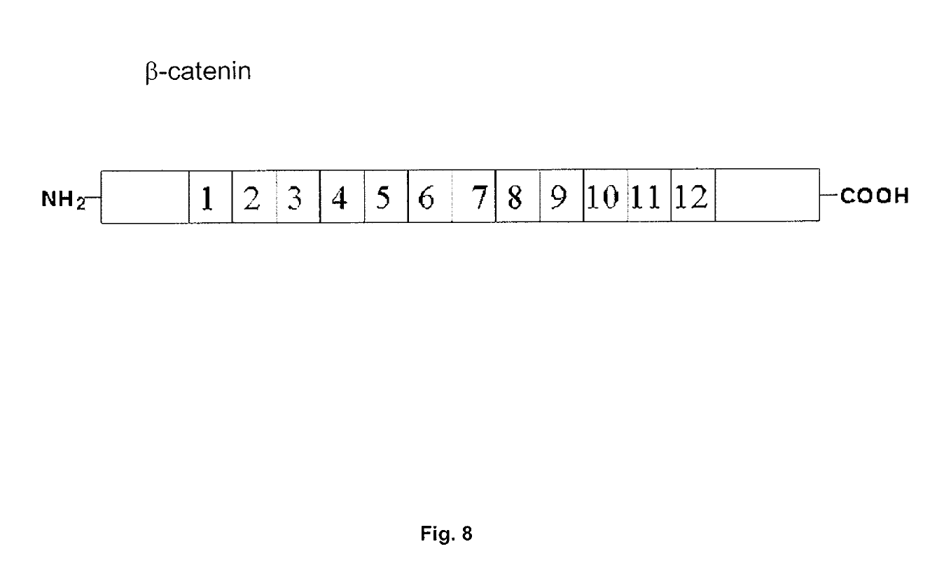

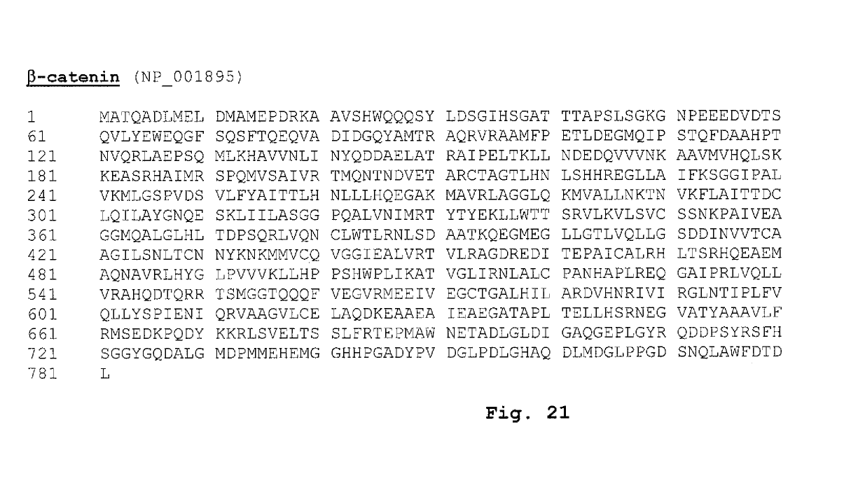

The domains of β-catenin involved in transcriptional activation have been localized in the N- and C-terminal parts of this molecule. Referring to FIG. 8, the N- and C-termini flank 12 armadillo-like repeat domains. The C-terminal tail of β-catenin, when fused to LEF-1, has been shown to be sufficient to promote transactivation. The N- and C-terminal transactivation domains of β-catenin interact with a growing list of nuclear factors that include the TATA-binding protein (TBP)1, Pontin, Teashirt, Sox17 and 13, histone deacetylase, SMAD4, the retinoic acid receptor, and the CREB binding protein and related proteins. FIG. 21 shows the amino acid sequence of human β-catenin.

### Lin28

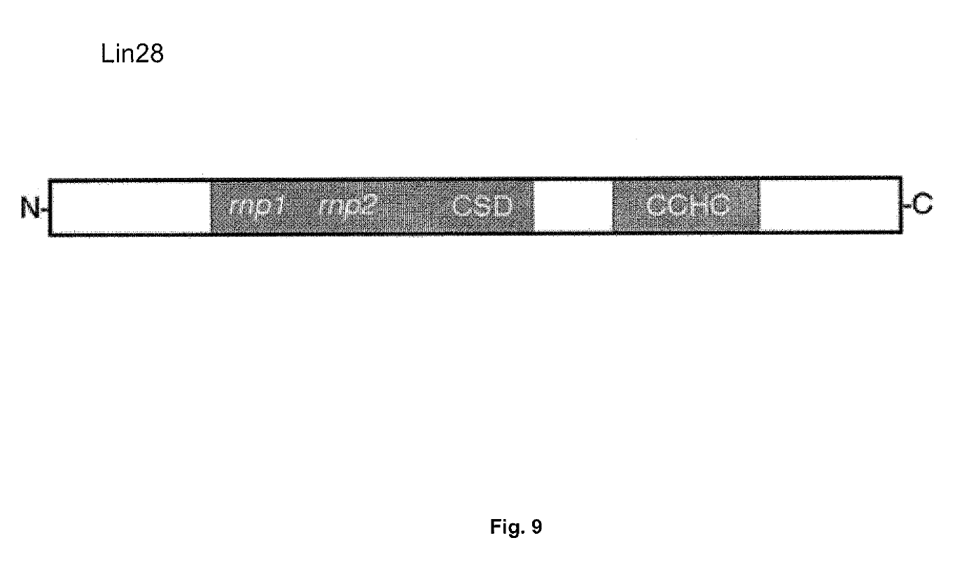

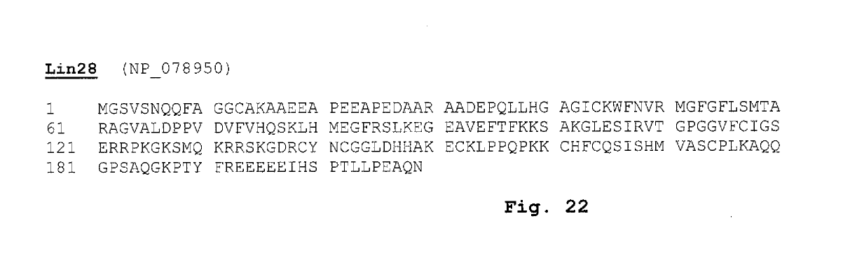

Lin28 is a conserved cytoplasmic protein with an unusual pairing of RNA-binding motifs (rnp1 and rnp2) in a cold shock domain (CSD) and a pair of retroviral-type CCHC zinc fingers (Balzer, E. and Moss E. G. 2007 RNA Biology 4:16-25). In the nematode C. elegans, it is a regulator of developmental timing. In mammals, it is abundant in diverse types of undifferentiated cells. In pluripotent mammalian cells, Lin28 is observed in RNase-sensitive complexes with Poly(A)-Binding Protein, and in polysomal fractions of sucrose gradients, suggesting it is associated with translating mRNAs. Upon cellular stress, Lin28 locates to stress granules, which contain non-translating mRNA complexes. However, Lin28 also localizes to cytoplasmic Processing bodies, or P-bodies, sites of mRNA degradation and microRNA regulation, consistent with it acting to regulate mRNA translation or stability. Mutational analysis shows that Lin28's conserved RNA binding domains cooperate to put Lin28 in mRNPs, but that only the CCHC domain is required for localization to P-bodies. When both RNA-binding domains are mutated, Lin28 accumulates in the nucleus, suggesting that it normally shuttles from nucleus to cytoplasm bound to RNA. Such studies are consistent with a model in which Lin28 binds mRNAs in the nucleus and accompanies them to ribosomes and P-bodies. Indeed, Lin28 has been shown to block the processing of pri-let-7 micro-RNAs in embryonic stem cells and act as a critical negative regulator in blocking miRNA-mediated differentiation of stem cells in certain cancers (Viswanathan, S. R. et al. 2008 Science 320:97-100). Lin28 may influence the translation or stability of specific mRNAs during differentiation. FIG. 9 is a schematic representation of the Lin28 protein and FIG. 22 shows the amino acid sequence of human Lin 28.

Enhancement of Protein iPSC Efficiency Using Epigenetic Modification

A great deal of recent research has concentrated on the epigenetic basis of pluripotency. It is suggested that differences between various cell types may be due to differences in global epigenetic profiles. DNA information content remains unchanged during differentiation. However, access to this information may become progressively more restricted as differentiation occurs. Epigenetic modifications of the genome may act as control access points to the DNA. Ectopic expression of reprogramming factors in fibroblasts may trigger a sequence of epigenetic events such as chromatin modifications or changes to DNA methylation necessary for the iPSC phenotype. Indeed, it has been recently found that the addition of the epigenetic drug BIX, an inhibitor of the G9a histone methyltransferase can improve the reprogramming efficiency in neural progenitor cells transduced with lentiviruses expressing Oct3/4 and Klf4 to a level comparable to that seen following lentiviral transduction with all 4 reprogramming factors (Oct3/4, Sox2, Klf4, and c-Myc). As such, the generation of iPSCs may also be carried out in the presence of a variety of small molecule chemical modulators (e.g., BIX) of histone modifying enzymes or in the presence of pluripotency factors (e.g., valproic acid) to augment the effects of the iPS inducing transcription machinery and thus increase the efficiency of iPSC generation following direct protein induction. Epigenetic modifying reagents, such as BIX and valproic acid, are available from commercial sources.

### Identification of Genes Involved in Maintenance of Embryonic Stem Cell Phenotype

A variety of methods can be used to identify genes that are associated with maintenance of the ES cell phenotype. In one embodiment, the expression level of genes in ES cells is compared to those in a somatic cell population. Candidate ES cell maintenance genes can be identified by quantifying and comparing the amounts of mRNAs or proteins expressed from the various genes in the ES and somatic cell populations. Candidate genes involved in the maintenance of the embryonic stem cell phenotype are expressed at a higher or lower level in ES cells as compared to the corresponding somatic cell.

Multiple techniques are known in the art to identify differences in mRNA expression between cell populations including DNA microarrays, differential display, nucleic acid subtraction, serial analysis of gene expression (SAGE), and Reverse Transcriptase-Polymerase Chain Reaction (RT-PCR). Differences in protein expression between cell populations can be determined, e.g., by antibody arrays and mass spectroscopy.

### DNA Microarrays

In some embodiments, transcripts are analyzed from the first longevity and wild type organisms. One method for comparing transcripts uses nucleic acid microarrays that include a plurality of addresses, each address having a probe specific for a particular transcript. Such arrays can include at least about 100, or about 1000, or about 5000 different probes, so that a substantial fraction, e.g., at least about 10%, 25%, 40%, 50%, or 75% of the genes in an organism are evaluated. mRNA can be isolated from a sample of the organism or from the whole organism. The mRNA can be reversed transcribed into labeled cDNA. The labeled cDNAs are hybridized to the nucleic acid microarrays. The arrays are detected to quantitate the amount of cDNA that hybridizes to each probe, thus providing information about the level of each transcript.

Methods for making and using nucleic acid microarrays are well known. For example, nucleic acid arrays can be fabricated by a variety of methods, e.g., photolithographic methods, mechanical methods (e.g., directed-flow methods), pin based methods, and bead based techniques. The capture probe can be a single stranded nucleic acid, a double-stranded nucleic acid (e.g., which is denatured prior to or during hybridization), or a nucleic acid having a single-stranded region and a double stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the nucleic acid. The Tₘ of the capture probe can be optimized by prudent selection of the complementarily region and length. Ideally, the Tₘ of all capture probes on the array is similar, e.g., within about 20° C., 10° C., 5° C., 3° C., or 2° C. of one another. A database scan of available sequence information for a species can be used to determine potential cross-hybridization and specificity problems.

The isolated mRNA from samples for comparison can be reversed transcribed and optionally amplified, e.g., by RT-PCR. The nucleic acid can be labeled during amplification, e.g., by the incorporation of a labeled nucleotide. Examples of preferred labels include fluorescent labels, e.g., red-fluorescent dye, Cy5 (Amersham) or green-fluorescent dye Cy3 (Amersham), and chemiluminescent labels. Alternatively, the nucleic acid can be labeled with biotin, and detected after hybridization with labeled streptavidin, e.g., streptavidin phycoerythrin (Molecular Probes).

The labeled nucleic acid can be contacted with the array. In addition, a control nucleic acid or a reference nucleic acid can be contacted with the same array. The control nucleic acid or reference nucleic acid can be labeled with a label other than the sample nucleic acid, e.g., one with a different emission maximum. Labeled nucleic acids can be contacted with an array under hybridization conditions. The array can be washed, and then imaged to detect fluorescence at each address of the array.

A general scheme for producing and evaluating profiles can include the following. The extent of hybridization at an address is represented by a numerical value and stored, e.g., in a vector, a one-dimensional matrix, or one-dimensional array. The vector x has a value for each address of the array. For example, a numerical value for the extent of hybridization at a first address is stored in variable xₐ. The numerical value can be adjusted, e.g., for local background levels, sample amount, and other variations. Nucleic acid is also prepared from a reference sample and hybridized to an array (e.g., the same or a different array), e.g., with multiple addresses. The vector y is constructed identically to vector x. The sample expression profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different nucleic acids detected by the array.

The expression data can be stored in a database. The database can have multiple tables. For example, raw expression data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

### Differential Display

Differential display is another well-established technique used to identify and isolate genes that are differentially expressed between two cell populations. In this approach, mRNA sequences from cell populations to be compared are reverse transcribed and amplified by PCR using a set of oligonucleotide primers, one anchored to the poly(A) tail and the other to a short arbitrary oligonucleotide that binds at varying distances from the poly(A) tail for the various RNA molecules. For some RNA molecules, the separation between the two primer sequences is too large to allow PCR amplification so that only a subset of RNA molecules are amplified. Separation of the amplified sequences on a DNA sequencing gel allows visualization of each of the amplified sequences. Comparison of gels for two cell populations reveals sequences that are abundant in one but not the other. Use of several different primer sets allows analysis of a larger number of genes. Sequences of interest may be excised from the gel and cloned. The advantages of differential display include its ease of use and its power to discover previously unknown differences. Its principal disadvantages are that not all differences are discovered using a single arbitrary primer, recovery of interesting DNA fragments is somewhat time consuming and differences in levels of expression are difficult to quantify. Nonetheless, this technique has been widely and successfully applied to analysis of human disease states.

### Nucleic Acid Subtraction

Subtraction techniques to clone differences between two mRNA populations are well developed. The process begins with reverse transcription of the mRNA from two populations to form cDNA. In one approach, the “driver” cDNA is labeled to allow affinity separation of the labeled driver sequences. The driver cDNA is then hybridized in excess to “tester” cDNA from the other population and the driver-driver and tester-driver hybrid molecules are removed by affinity separation. Alternately, the driver cDNA and hybrid molecules are enzymatically removed by digestion with exonucleases rather than by physical partitioning.

### Serial Analysis of Gene Expression (SAGE)

The relative frequency of gene expression can also be determined by sequencing a large number of cDNA fragments in a library prepared from the cells or tissue of interest. This is accomplished by ligating together short ˜10 bp long sequence “tags” from the 3′-most NlaIII restriction sites of multiple genes. The tags are separated by distinctive linker sequences so the various sequences can be distinguished. The ligated sequences from many different concatimers are then sequenced and the results compiled to form a distribution showing the frequencies of the various gene-associated tags. This process is sufficiently efficient that from about 10⁴ to about 10⁵ tags can be sequenced from each library. The main advantage of SAGE is its unbiased assessment of the frequencies with which genes are expressed. Disadvantages include the lack of clones from novel tags that may appear during sequencing and the need for extensive sequencing to accurately assess levels of expression of weakly expressed genes.

### Reverse Transcriptase Polymerase Chain Reaction (RT-PCR)

The most sensitive quantitative method to compare mRNA levels in different sample populations is RT-PCR. The first step is the isolation of mRNA from a target sample. The starting material is typically total RNA isolated from test cells or tissues and corresponding control cell or tissues. General methods for mRNA extraction are well known in the art and are disclosed in standard textbooks of molecular biology, including Ausubel et al., Current Protocols of Molecular Biology, John Wiley and Sons (1997). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns.

As RNA cannot serve as a template for PCR, the first step in gene expression profiling by RT-PCR is the reverse transcription of the RNA template into cDNA, followed by its exponential amplification in a PCR reaction. Two commonly used reverse transcriptases are Avian Myeloblastosis Virus Reverse Transcriptase (AMV-RT) and Moloney Murine Leukemia Virus Reverse Transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

A variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR.

Although the PCR step can use a variety of thermostable DNA-dependent DNA polymerases, it typically employs the Taq DNA polymerase, which has a 5′-3′ nuclease activity, but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution and signal from the released reporter dye is free from the quenching effect of the second fluorophore. One molecule of reporter dye is liberated for each new molecule synthesized, and detection of the unquenched reporter dye provides the basis for quantitative interpretation of the data.

Once a set of nucleic acid transcripts are identified as being associated with aging or lifespan regulation, it is also possible to develop a set of probes or primers that can evaluate a sample for such markers. For example, a nucleic acid array can be synthesized that includes probes for each of the identified markers.

### Protein Analysis

The abundance of a plurality of protein species can be determined in parallel, e.g., using an array format, e.g., using an array of antibodies, each specific for one of the protein species. Other ligands can also be used. Antibodies specific for a polypeptide can be generated by known methods.

Methods for producing polypeptide arrays are known in the art. For example, a low-density (96 well format) protein array can be used in which proteins are spotted onto a nitrocellulose membrane. A high-density protein array for antibody screening may be formed by spotting proteins onto polyvinylidene difluoride (PVDF). Polypeptides can be printed on a flat glass plate that contained wells formed by an enclosing hydrophobic Teflon mask. Also, polypeptide can be covalently linked to chemically derivatized flat glass slides in a high-density array (about 1600 spots per square centimeter). Investigators have described a high-density array of 18,342 bacterial clones, each expressing a different single-chain antibody, in order to screening antibody-antigen interactions. These art-known methods and other can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address and analyze similar to nucleic acid arrays.

### Mass Spectroscopy

Mass spectroscopy can also be used, either independently or in conjunction with a protein array or 2D gel electrophoresis. For 2D gel analysis, purified protein samples from the ES cells and somatic cells are separated on 2D gels (by isoelectric point and molecular weight). The gel images can be compared after staining or detection of the protein components. Then individual “spots” can be proteolyzed (e.g., with a substrate specific protease, e.g., an endoprotease such as trypsin, chymotrypsin, or elastase) and then subjected to MALDI-TOF mass spectroscopy analysis. The combination of peptide fragments observed at each address can be compared with the fragments expected for an unmodified protein based on the sequence of nucleic acid deposited at the same address. The use of computer programs (e.g., PAWS) to predict trypsin fragments, for example, is routine in the art. Thus, each address of spot on a gel or each address on a protein array can be analyzed by MALDI-TOF mass spectroscopy. The data from this analysis can be used to determine the presence, abundance, and often the modification state of protein biomolecules in the original sample. Most modifications to proteins cause a predictable change in molecular weight.

Other methods can also be used to profile the properties of a plurality of protein biomolecules. These include ELISAs and Western blots. Many of these methods can also be used in conjunction with chromatographic methods and in situ detection methods (e.g., to detect subcellular localization).

### Proteins and Peptides

The present invention relates to isolated and/or recombinant (including, e.g., essentially pure) proteins or polypeptides, which are able to induce and maintain the ES cell phenotype. Proteins or polypeptides referred to herein as “isolated” are proteins or polypeptides purified to a state beyond that in which they exist in mammalian cells. Isolated” proteins or polypeptides include proteins or polypeptides obtained by methods described herein, similar methods or other suitable methods, including essentially pure proteins or polypeptides, proteins or polypeptides produced by chemical synthesis, or by combinations of biological and chemical methods, and recombinant proteins or polypeptides which are isolated. Proteins or polypeptides referred to herein as “recombinant” are proteins or polypeptides produced by the expression of recombinant nucleic acids of the present invention.

The invention also relates to isolated and/or recombinant portions or fragments of a proteins or polypeptides that are able to induce and maintain the ES cell phenotype. In one embodiment, an isolated and/or recombinant portion (e.g., a peptide) has at least one function characteristic of a human protein or polypeptide, which is able to induce and maintain the ES cell phenotype, such as a binding function. Examples of functional fragments or portions of a proteins or polypeptides which are able to induce and maintain the ES cell phenotype include those with deletions of one or more amino acids from the mature protein which retain one or more functions. The amino acids which can be deleted can be identified by screening. For example the N- or C-terminus of the protein can be deleted in a step-wise fashion and the resulting protein or polypeptide screened in one or more assays as described herein. Also envisioned are fragments wherein an (i.e., one or more) internal amino acid is deleted, including deletions of non-contiguous amino acids. Where the resulting protein displays activity in the assay, the resulting protein (“fragment”) is functional.

Studies on the structure and function of proteins or polypeptides which are able to induce and maintain the ES cell phenotype provide the basis for being able to divide such proteins or polypeptides into functional domains (e.g., HMG DNA binding domain, leucine zipper, leader peptide, mature protein). Portions of human proteins or polypeptides which are able to induce and maintain the ES cell phenotype can be produced which have full or partial function on their own, or which when joined with another portion of a second protein of interest.

The invention further relates to mutants, variants or derivatives of a human protein or polypeptide that is able to induce and maintain the ES cell phenotype. Such variants include natural or artificial variants, differing by the addition, deletion or substitution of one or more amino acid residues, or modified polypeptides in which one or more residues is modified, and mutants comprising one or more modified residues.

The invention further relates to fusion proteins, comprising a human proteins or polypeptides which are able to induce and maintain the ES cell phenotype as a first moiety, linked to a second moiety not occurring in nature. Thus, the second moiety can be an amino acid or polypeptide. The first moiety can be in an N-terminal location, C-terminal location or internal to the fusion protein. In one embodiment, the fusion protein comprises a human protein or polypeptide which is able to induce and maintain the ES cell phenotype or portion thereof as the first moiety, and a second moiety comprising a linker sequence and affinity ligand (e.g., an enzyme, an antigen, epitope tag).

Fusion proteins can be produced by a variety of methods. For example, some embodiments can be produced by the insertion of human proteins or polypeptides which are able to induce and maintain the ES cell phenotype gene or portion thereof into a suitable expression vector, such as Bluescript®II SK +/− Stratagene), pGEX-4T-2 (Pharmacia) and pET-15b (Novagen). The resulting construct is then introduced into a suitable host cell for expression. Upon expression, fusion protein can be isolated or purified from a cell lysate by means of a suitable affinity matrix (see e.g., Current Protocols in Molecular Biology (Ausubel, F. M. et al., eds., Vol. 2, Suppl. 26, pp. 16.4.1-16.7.8 (1991)).

### Delivery of Protein to Cells

Some embodiments relate to a method of de-differentiating somatic cells to an embryonic stem cell state comprises delivery into said somatic cell an isolated protein, wherein the protein effects de-differentiation of said somatic cell to an embryonic stem cell phenotype.

Some embodiments relate to a method of de-differentiating somatic cells to an embryonic stem cell state comprises delivery into the nucleus of said somatic cell an isolated protein selected from the group consisting of Oct3/4, Sox2, Nanog, Stat3, Eras, c-Myc, Klf4, β-catenin and Lin28, wherein the protein effects de-differentiation of said somatic cell to an embryonic stem cell phenotype.

Some embodiments relate to a method of de-differentiating somatic cells to an embryonic stem cell state comprises delivery into the nucleus of said somatic cell an isolated protein molecule that comprises an amino acid sequence selected from SEQ ID NOs: 1-12, wherein the protein effects de-differentiation of said somatic cell to an embryonic stem cell phenotype.

In some embodiments, the amino acid molecule delivered into the nucleus of the somatic cell has at least 50%, 60%, 65%, 70%, 75%, 80%, 85%, 90%, 95%, or 98% sequence identity to an amino acid sequence (e.g., to the entire length of the amino acid sequence) including SEQ ID NOs: 1-12.

In other embodiments, the amino acid delivered is an isolated fragment or portion of an amino acid molecule comprising an amino acid selected from SEQ ID NOs: 1-12. In some embodiments, the fragment is at least 10, 15, 20, 25, 30, 50, 75, 100, 150, 200 or more amino acids in length.

Proteins of interest can be introduced into cells by traditional methods such as lipofection, electroporation, calcium phosphate precipitation, particle bombardment and/or microinjection.

Delivery of molecules by exposing cells to pulses of laser beam (laserfection or laser transfection) has also been described, as have delivery by pinocytosis or use of streptolysin-O (SLO). As another example, a kit from Active Motif utilizing the PEP-1 peptide as a delivery system for proteins ranging from a small peptide to a large IgG antibody is commercially available (Chariot™, see activemotif.com on the world-wide web). However, these methods require manipulation of the cells, e.g., adding and removing transfection materials, pre-treating cells, and special apparatus and equipment, etc.

While the general methods above are suitable for introducing molecules into cells, other methods of introducing proteins of interest into the cell may be used. For example, proteins can be coupled to the HIV TAT sequence, which most cells naturally uptake. The chimeric probe can simply be, e.g., added to cell culture or injected into the animal for delivery.

The proteins of interest are optionally associated (covalently or non-covalently) with a cellular delivery module that can mediate its introduction into the cell. The cellular delivery module is typically, but need not be, a polypeptide, for example, a nuclear localization sequence (NLS), a PEP-1 peptide, an amphipathic peptide, e.g., an MPG peptide, a cationic peptide (e.g., a homopolymer of lysine, histidine, or D-arginine), or a protein transduction domain (a polypeptide that can mediate introduction of a covalently associated molecule into a cell). For example, a protein of interest can be covalently associated with a protein transduction domain (e.g., an HIV TAT sequence, which most cells naturally uptake, or a short D-arginine homopolymer, e.g., 8-D-Arg, eight contiguous D-arginine residues). The protein transduction domain can be covalently attached directly to the protein of interest, or can be indirectly associated with the sensor (for example, the protein transduction domain can be covalently coupled to a bead or to a carrier protein such as BSA, which is in turn coupled to the sensor, e.g., through a photolabile or cleavable linker. The protein transduction domain-coupled protein of interest can simply be, e.g., added to cell culture or injected into an animal for delivery.

A nuclear localizing sequence (NLS) is an amino acid sequence that is exposed on surface of a protein. NLS are recognized by cytosolic nuclear transport receptors, which transport proteins into the cell nucleus through the Nuclear Pore Complex. Typically, a NLS sequence consists of one or more short sequences of positively charged lysines or arginines.

A number of polypeptides capable of mediating introduction of associated molecules into a cell are known in the art and can be adapted to the present invention. See, e.g., Langel (2002) Cell Penetrating Peptides CRC Press, Pharmacology & Toxicology Series.

The proteins of interest can also be introduced into cells by covalently or noncovalently attached lipids, e.g., by a covalently attached myristoyl group. Lipids used for lipofection are optionally excluded from cellular delivery modules in some embodiments.

In some embodiments, proteins of interest are delivered to cells using Profect protein delivery reagents as described below in Example 1.

The cell into which a protein of interest is introduced can be a mammalian cell (e.g., a human cell). The cell can be, e.g., in culture or in a tissue, fluid, etc. and/or from or in an organism.

### Example 1

### Profect Protein Delivery

### Reagents

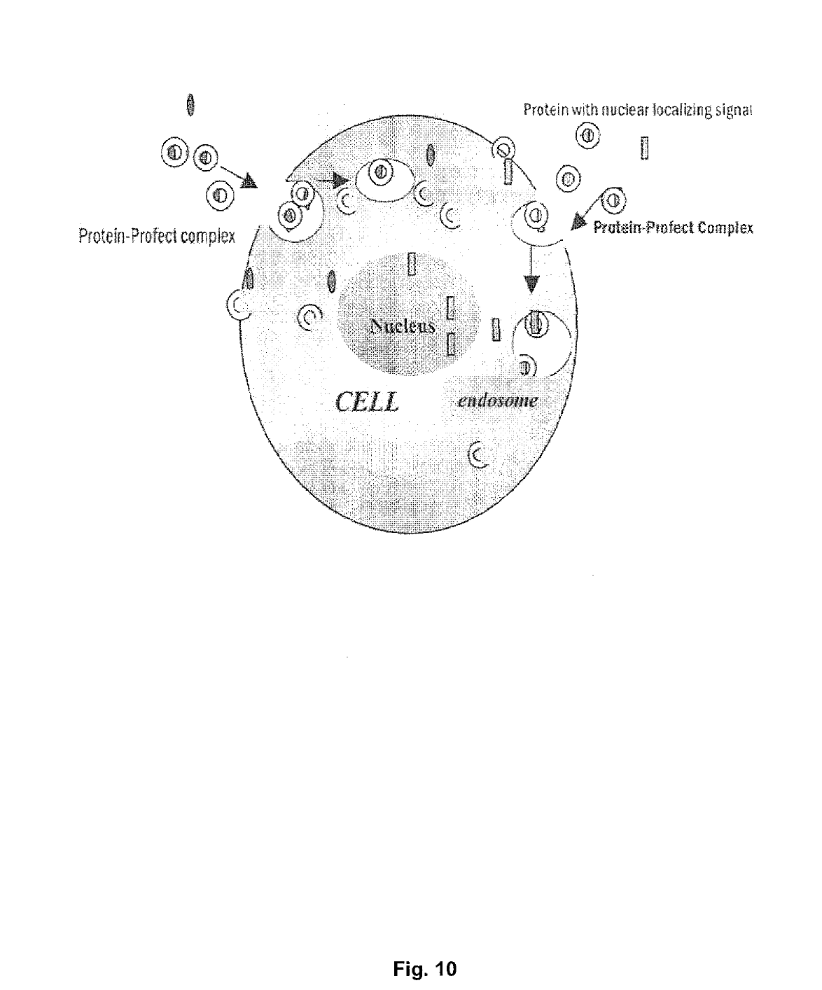

Profect protein delivery reagents are available from Targeting Systems, El Cajon, Calif., accessible on the world-wide-web at targetingsystems.com. Profect-P1 is a lipid reagent that forms non-covalent complexes with proteins and enables translocation of intact functional proteins across the cell membrane. Profect-P2 is a non-lipid reagent that forms non-covalent complexes with proteins and enables protein transport across both the cell membrane as well as the nuclear membrane. Profect-P2 has endosomolytic properties which protect the internalized protein from being degraded in the lysosomes. Profect-P2 also has the unique ability to escort both DNA and protein across the nuclear membrane. Profect-P1 and Profect-P2 can form non-covalent complexes with a variety of proteins and can be used to successfully co-deliver different proteins. Proteins delivered with Profect range from 10 KDa to 540 KDa. Referring to FIG. 10, Intracellular protein delivery occurs as a result of fusion of the Protein-Profect-P1 complexes with the cell membrane or endocytosis of the Protein-Profect-P1/P2 complexes. Endosomal lysis mediated by Profect results in release of the protein in the intracellular environment.

The most important property of the Profect reagents is that they enable highly efficient delivery of intact, functional proteins into many difficult-to-transfect primary cell types and several cell lines. Versatility: These reagents have been used to successfully deliver a variety of proteins (11,000 Kd to 540,000 Kd) into a variety of primary cell lines. Compatibility with Cell culture media and antibiotics: The reagents are compatible with transfection to physiological buffer such as those involving signal transduction cannot be carried out in OptiMEM1 or media with growth factors as these influence signal transduction

### Site-Specific Protein Delivery

The Profect reagents provide a mechanism for site-specific protein delivery (e.g., nuclear delivery) in many instances. This is important in cases where it is desirable to target the protein to a desired sub cellular organelle. Nuclear delivery is effected by using the Profect P2 reagent and made more efficient by co-delivering the protein (e.g., IgG) with histone to target the nucleus. Similarly targeting to other organelles can often be accomplished by co-delivering the protein of interest with a protein that localizes to the organelle of interest.

The ability of Profect reagents to mediate efficient protein transfection was first tested using β-galactosidase (540 Kd) as a reporter protein. In these experiments, 100 ng of 3-galactosidase was complexed with 5 μl of the Profect reagent (Profect-P1 or P2) in 500 μl of PBS and used to transfect cells in 12-well dishes. The cells were exposed to the protein-Profect complexes for 1 hr at 37° C., then washed twice with PBS, fixed and stained for visualization of β-galactosidase activity. All 4-cell types tested (NIH 3T3, HeLa, retinal pigmented epithelial cells and human lens epithelial cells) showed efficient delivery of β-galactosidase (85-100%)

An important requisite for versatile application of a protein delivery reagent is the ability to control the amount of protein delivered into the cells. To test this, HeLa cells were transfected with either 600 ng or 3 μg of β-galactosidase using the Profect-P2 reagent. 100% of cells were transfected with the β-galactosidase protein. Cells transfected with 3 β-galactosidase showed higher activity than cells transfected with 600 ng β-galactosidase indicating that it is possible to control amount of protein delivered into cells by manipulating the amount of protein used for transfection.

The Profect reagents can deliver functional, intact proteins. An important requisite for an efficient protein delivery systems is that proteins delivered intracellularly should retain their normal physiological functions. In an effort to demonstrate the efficacy of the Profect reagents to deliver, CV-1 cells and MCF-7 cells were transfected with active caspase 3 and examined the cells for apoptosis using phase contrast microscopy combined with DAPI staining (in case of CV-1 cells) or assessed apoptosis with the help of the Vybrant apoptosis assay kit (in case of MCF-7 cells). In these experiments the caspase-transfected CV-1 cells were also stained with the nuclear stain DAPI and examined by fluorescence microscopy to assess condensation and fragmentation of the nucleus that is a characteristic of caspase induced apoptosis. MCF-7 cells transfected with caspase were exposed for 30 minutes to a combination of two dyes (Yopro, and propidium iodide) in the vybrant apoptosis assay kit. Approximately 3-4 hrs post-transfection and then examined by fluorescence microscopy. The results of this experiment suggest that transfection with active caspase 3 showed extensive cell death together with condensed, fragmented nuclei, whereas cells transfected with a control protein (β-galactosidase) using the Profect-P2 reagent show intact nuclei and healthy cells. The results of the apoptosis experiment in MCF-7 cells showed that MCF-7 cells transfected with active caspase using Profect-P2 showed extensive apoptosis whereas cells transfected with caspase in the absence of Profect did not show any fluorescence.

### Profect Protocol

Profect-P1 reagent is Vortexed at full speed for 30 seconds just before use. Profect-P1 reagent is stored at −20° C. The Profect-P2 reagent can be stored at 4° C. or at −20° C. An exemplary protocol is as follows:

1. Set up cells to be transfected in Labtek-chamber slides so that they are about 80% confluent at the time of the experiment.

2. Add 0.5-5 μl of protein solution (100 ng to 10 μg, in general we recommend 5 μg) to a sterile tube containing the appropriate amount of serum-free DMEM.

3. Add 3 μl or 5 μl of Profect reagent (mix well before use).

4. Gently mix the transfection complex mixture by flicking the tube.

5. Incubate at room temperature for 20 minutes.

6. Remove serum-containing growth media from cells by aspirating, wash cells with serum-free medium and add 1 ml of serum-free medium to each well.

7. Add the transfection complex mixture to cells.

8. Return plate to incubator and incubate for 2-5 hours.

9. Add 1 ml of complete media (containing 10% serum) to each well.

10. Replace media on the following day and continue incubation until assaying. Wash cells with serum-free medium before assaying to remove any untransfected protein.

The transfection complex mixture is composed of protein and Profect Transfection Reagent in serum-free medium. For example, at the incubation step (step 8) (6-well format), transfection complex mixture consisting of 2 μg protein, and 3 μl Profect Transfection Reagent in 200 μl serum-free medium is added to a well containing cells in a 1 ml volume.

### Peptide Delivery

6.5 μg peptide is mixed well with 5 μl of the P-2 reagent in 100 μl of high glucose DMEM. The peptide/Profect mixture is incubated at room temperature for 20 minutes then Vortex for 15 seconds. The complexes are diluted to 1 ml with high glucose DMEM prior to following the transfection protocol above. For 96 well plates, the peptide/Profect is mixed well and 40 μl of complex are added per well of a 96 well plate (aspirate culture media before addition of transfection complex). Following incubation at 37° C. for 3 hrs, 100 μl of complete media is added followed by continued incubation. Cells are washed 4 times with serum free media and assay.

After the incubation period, the transfection complexes can be washed off by washing cells extensively (4 times with DMEM and assessing protein delivery without fixing the cells). Alternatively, the transfection complexes can be aspirated at the end of the incubation period and complete media added, waiting longer to assess effects of protein delivery on the cells. Serum-Free, high glucose OMEM may be used in place of OptiMem 1 to increase cell survival. OMEM can also be used as complexing medium for other applications.

### Example 2

### Identification of Embryonic Stem Cell Markers in Induced Pluripotent Stem Cells by DNA Microarray Analysis

Using a genetic approach, induced pluripotent stem (iPS) cells were generated from adult human dermal fibroblasts (HDF) by retroviral-mediated transduction of four transcription factors, namely Oct3/4, Sox2, Klf4, and c-Myc (Takahashi K. et al. 2007 Cell 131:861-872). The human iPS cells were similar to human embryonic stem (ES) cells in morphology, proliferation, surface antigens, gene expression, epigenetic status of pluripotent cell-specific genes, and telomerase activity.

DNA microarray analyses showed that the global gene-expression patterns are similar, but not identical, between human iPS cells and hES cells. Among 32,266 genes analyzed, 5,107 genes showed more than 5-fold difference in expression between HDF and human iPS cells (See Tables S3 and S4 of Takahashi, K. et al. 2007, supra), whereas 6083 genes between HDF and hES cells showed >5-fold difference in expression (See Tables S5 and S6 of Takahashi, K. et al. 2007, supra). In contrast, a smaller number of genes (1,267 genes) showed >5-fold difference between human iPS cells and hES cells (See Tables S7 and S8 of Takahashi, K. et al. 2007, supra).

### Example 3

### Identification of Candidate Reprogramming Factors

To identify candidate reprogramming factors, Yu et al. (2007 Science 318:1917-1920) compiled a list of genes with enriched expression in human ES cells relative to that of myeloid precursors and prioritized the list based on known involvement in the establishment or maintenance of pluripotency (Table 1). The investigators showed that, of these, four factors (Oct4, Sox2, Nanog and Lin28) were sufficient to reprogram human somatic cells to pluripotent stem cells that exhibit the essential characteristics of embryonic stem (ES) cells.

TABLE 1
List of Human ES cell-enriched genes

| Gene | Accession Number |
|---|---|
| POU5F1 (Oct3/4) | NM_002701 |
| NANOG | NM_024865 |
| SOX2 | NM_003106 |
| FOXD3 | NM_012183 |
| UTF1 | NM_003577 |
| DPPA3 | NM_199286 |
| ZFP42 | NM_174900 |
| ZNF206 | NM_032805 |
| SOX15 | NM_006942 |
| PHB | NM_002634 |
| MYBL2 | NM_002466 |
| LIN28 | NM_024674 |
| BCL2 | NM_000633 |
| DPPA2 | NM_138815 |
| DPPA4 | NM_018189 |
| DPPA5 | NM_001025290 |
| DNMT3B | NM_006892 |
| DNMT3L | NM_013369 |
| GBX2 | NM_001485 |
| TERF1 | NM_017489 |
| HESX1 | NM_003865 |
| SALL4 | NM_020436 |
| SALL1 | NM_002968 |
| SALL2 | NM_005407 |
| SALL3 | NM_171999 |
| TDGF1 | NM_003212 |
| GDF3 | NM_020634 |
| NODAL | NM_018055 |
| LIN28B | NM_001004317 |
| MGC27016 | NM_144979 |
| PRDM14 | NM_024504 |
| USP44 | NM_032147 |
| PHC1 | NM_004426 |
| PIWIL2 | NM_018068 |
| POU3F2 | NM_005604 |
| POU6F1 | NM_002702 |
| NPM2 | NM_182795 |
| NPM3 | NM_006993 |
| ACRBP | NM_032489 |
| AKT | NM_005163 |
| C10orf96 | NM_198515 |
| C14or115 | NM_018228 |
| C9orf135 | NM_001010940 |
| CCNF | NM_001761 |
| CER1 | NM_005454 |
| CLDN6 | NM_021195 |
| CTSL2 | NM_001333 |
| DDX25 | NM_013264 |
| DKFZp761P0423 | XM_291277 |
| ECAT1 | NM_001017361 |
| ECAT11 | NM_019079 |
| ECAT8 | XM_117117 |
| EMID2 | NM_133457 |
| FLJ35934 | NM_207453 |
| FLJ40504 | NM_173624 |
| FLJ43965 | NM_207406 |
| FOXH1 | NM_003923 |
| GAP43 | NM_002045 |
| GPC2 | NM_152742 |
| GPR176 | NM_007223 |
| GPR23 | NM_005296 |
| HES3 | NM_001024589 |
| HRASLS5 | NM_054108 |
| LHX5 | NM_022363 |
| LIN41 | NM_001039111 |
| LOC138255 | NM_001010940 |
| LOC389023 | BC032913 |
| LOC643401 | BC039509 |
| MDK | NM_001012334 |
| MIRH1 | XM_931068 |
| MIXL1 | NM_031944 |
| NHLH2 | NM_005599 |
| NR0B1 | NM_000475 |
| NUT | NM_175741 |
| OTX2 | NM_172337 |
| PRTG | NM_173814 |
| PUNC | NM_004884 |
| RABGAP1L | NM_014857 |
| RKHD3 | NM_032246 |
| RPGRIP1 | NM_020366 |
| SCGB3A2 | NM_054023 |
| SLITRK1 | NM_052910 |
| SOX10 | NM_006941 |
| SOX11 | NM_003108 |
| SOX21 | NM_007084 |
| SP8 | NM_198956 |
| SPANXC | NM_022661 |
| SYT6 | NM_205848 |
| T | NM_003181 |
| TCL1A | NM_021966 |
| TDRD5 | NM_173533 |
| TSGA10IP | NM_152762 |
| UNC5D | NM_080872 |
| ZNF124 | NM_003431 |
| ZNF342 | NM_145288 |
| ZNF677 | NM_182609 |
| ZNF738 | BC034499 |

### Example 4

### Generation of iPSCs from Fibroblasts Using a Novel Protein Delivery Tool

### Recombinant Protein(s)

Reprogramming transcription factors are purchased from commercial sources when and where available. Non-commercially available proteins are expressed in E. coli using the pET expression system (Novagen) and purified (e.g., using either His- or GST-binding columns). Full-length Nanog (Peprotech) and Sox2 (Abnova) were obtained commercially. We obtained both an GST-human POU5F1 (Oct3/4) expression plasmid (from Dean Tantin, University of Utah School of Medicine) and an pET28C-Human Lin-28 expression plasmid (from Eric G. Moss, University of Medicine and Dentistry of New Jersey). Overnight cultures of expression plasmids grown in BL21-DE3 are diluted in LB (1:20), grown to an OD660 of 0.5-0.6 and induced with 1 mm IPTG for 4 hours at 30 C. Cells are lysed and the recombinant produced proteins purified using either the Bugbuster GST bind or Bugbuster His bind Purification Kits (Novagen Cat #70794-3). If the presence of the GST tag or the His tag fusion partner is found to hinder the nuclear localization of the recombinant protein, the fusion partner is cleaved using the protease thrombin and the Thrombin Cleavage Capture Kit (Novagen Cat#69022). Fortuitously, both Oct-4 and Lin 28 expression constructs both contain a vector encoded thrombin cleavage site immediately upstream of the target proteins and both full length Oct-4 and Lin-28 protein sequences lack any potential native thrombin cleavage sites.

### Nuclear Targeting

It is not widely known that proteins can be transfected into living cells, much like DNA. As disclosed herein, recombinant proteins are targeted to the nuclei of human fibroblasts using Profect Protein Delivery System (Targeting Systems, Santee, Calif., on the world-wide web at targetingsystems.com). The most important property of the Profect reagents is that they enable highly efficient delivery of intact, functional proteins into many difficult-to-transfect primary cell types and several cell lines. These reagents have been used to successfully deliver a variety of proteins (11 Kd to 540 Kd) into a variety of primary cell lines. The Profect reagents provide a mechanism for site-specific protein delivery (e.g., nuclear delivery) in many instances. This is important in cases where it is desirable to target the protein to a desired sub-cellular organelle. Nuclear delivery is effected by using the Profect P2 reagent and may be made more efficient by co-delivering the protein with histone to target the nucleus. Profect P2 is a non-lipid reagent that forms non-covalent complexes with proteins and enables protein transport across both the cell membrane as well as the nuclear membrane. In addition Profect P-2 has endosmolytic properties, which protect the internalized protein from being degraded in lysosomes. Proteins delivered intracellularly with this system have been shown to retain their normal physiological functions. A number of investigators have used the Profect Reagent (Nandan, D. et al. 2002 J Biol Chem 277:50190-50197; Sendide, K. et al. 2005 J Immunol 175:5324-5332; Miao, E. A. et al. 2006 Nature Immunology 7:569-575; Tanaka H. et al. 2006 Stem Cells 24:2592-2602; Soualhine H. et al. 2007 J Immunol 179:5137-5145).

### Protein Transfection

Human dermal fibroblasts are grown as monolayers in DMEM/F12 culture medium supplemented with non-essential amino acids, L-glutamine and 10% fetal bovine serum. Human fibroblasts in 6-well plates (80% confluent) are transfected with recombinant Oct3/4, Sox2, Nanog and Lin-28 and combinations there of as follows: 2 μg of total protein (0.5-5 μl) is added to a sterile tube containing 200 μl serum-free medium. 3 μl of Profect P2 reagent is gently added to the transfection complex mixture by gently flicking the tube. Meanwhile, serum-containing growth media is removed from the fibroblast cells by aspiration, the cells are washed with serum-free medium and 1 ml of serum-free medium added to each well. Following incubation of the Protein-Profect P2 transfection complexes at room temperature for 20 minutes, the transfection complex mixture consisting of 2 μg protein, and 3 μl Profect Transfection Reagent in 200 μl serum-free medium is added to a well containing cells in a 1 ml volume. Cells are returned to the incubator and the transfection complex is either removed or diluted out and replaced with complete media (containing 10% serum) 1-2 hours later.

Human dermal fibroblasts are transfected with the reprogramming factors every other day or as needed upon which the transfected fibroblasts are split and plated on irradiated mouse embryonic fibroblasts (iMEFs). Although reports have detailed the requirement of the factors to be expressed during lentiviral transduction for a period of 12 days to induce the iPSCs, this requirement may be specific to lentiviral transduction as we know that somatic cells can be reprogrammed to a pluripotent embryonic stem cell state during somatic cell nuclear transfer (SCNT) experiments.

The transfected human fibroblasts on MEFs are then subjected to culture in human embryonic stem (hES) cell conditions by changing the media to DMEM/F12 culture medium supplemented with KnockOut serum replacer, non-essential amino acids, L-glutamine, β-mercaptoethanol and basic fibroblast growth factor (bFGF).

iPSC Derivation

Cells are monitored daily for the appearance of colonies with human ES cell morphology (iPS colonies). Colonies are picked for expansion on day 20+ post-transduction. Human iPSCs are maintained on irradiated mouse embryonic fibroblasts (MEF) as above. Feeder-free culture on matrigel with conditioned medium may also be carded out as previously described. Passaging, expansion, and cryopreservation are carried out as previously described.

### iPS Characterization

Standard G-banding chromosome analysis is carried out. Telomerase activity is also assessed regularly. The immunocytochemical double-labeling technique initially examines OCT3/4 and NANOG, followed by the cell surface epitope SSEA-3. In situ staining of cells in culture provides staining information, which can be interpreted in light of the morphology and appearance of the colonies.

For flow cytometry, adherent cells are individualized by trypsin treatment and processed directly for antibody staining (CD133, CD9, Tra-1-81). Control samples are stained with isotype-matched control antibodies. Cells are analyzed on a FACSCalibur flow cytometer. 7-aminoactinomycin D is added before analysis for dead cell exclusion.

For quantitative RT-PCR total RNA is prepared as described with the RNeasy Mini Kit with on-column DNase I digestion. Quantitative PCR reactions are carried out with Power SYBRGreen® PCR Master Mix. The cDNA from human H1 ES cells is used as a relative standard for GAPDH, OCT4 and NANOG. The expression of genes of interest is normalized to that of GAPDH in all samples.

iPS samples are tested for embryoid body formation, established PSC markers, genome wide gene expression, miRNA profiles and SNP fingerprints. Gene microarray is currently the most accessible, comprehensive, and reliable technology for global gene expression analysis. For example, a platform by Illumina, Inc. uses 50mers for its bead-based platform. Functional genomics assays and standard assays for determining pluripotence in human cells are used. Our analysis of a large data set of >1000 microarray experiments, has led to the following observations: 1) sets of interacting genetic elements can codify pluripotent stem (PS) cell phenotypes; 2) microarray data can be used to generate robust models for pluriopotent types; and 3) these models allow for the a priori prediction of the properties of newly derived or iPSCs.

Gene microarray data are used to compare the iPSCs induced by protein transfection with lentiviral iPSCs and ePSC lines, as we have done with other PS cell lines. Systems biology approaches are applied to the resulting data (Mueller, et al. 2008 Nature 455:401-405). In these previous studies, we created and analyzed a stem cell-centric database of global gene expression profiles that enabled the classification of cultured human stem cells in the context of a wide variety of pluripotent, multipotent, and differentiated cell types. This database already contains the profile for human neural SC-30 cells and others. We are in a unique position to have in our National Human Neural Stem Cell Resource (NHNSCR), both human neural stem cell lines and matching fibroblast cells from the same patients. The expression profiles of SC-30 neural stem cells (NSCs) are compared with neural stem cells derived from the iPSCs induced here using the SC-30 fibroblasts obtained from the identical patient. These NSCs are derived using the same methodologies described below in Example 7.

SNP genotyping is used to provide unambiguous identification of iPSC lines, and to monitor genomic integrity by detecting variations that frequently occur in culture, including genomic duplications and deletions, and loss of heterozygosity. The Infinium® assay developed by Illumina, Inc. uses a method of sample preparation that makes it possible to read out any number of SNPs from one sample, limited only by the number of elements present on the microarray. On every array, each bead type is present an average of 15-30 times. This redundancy produces exquisite accuracy in calling of genotypes. Since these high-throughput methods monitor hundreds to hundreds of thousands of SNP variations, identification of the genome under investigation is an automatic byproduct of these highly multiplexed genotyping assays. In addition to measuring genomic abnormalities, high-density SNP arrays provide comprehensive information regarding the genetic profile of each stem cell analyzed.

### Example 5

### Enhancement of Direct Protein iPSC Efficiency Using Epigenetic Modification

Epigenetics refers to all heritable and potentially reversible changes in gene or genome functioning that occur without altering the nucleotide sequence of the DNA. A range of enzyme mediated modifications of chromatin (e.g., histone acetylation and methylation and chromatin remodeling) can activate or repress gene expression. It has become increasingly evident that epigenetic changes play an important role both in the maintenance of the pluripotent state and also in the ability of the reprogramming factors to induce the iPSC state. Ectopic expression of reprogramming factors is thought to perhaps trigger a sequence of epigenetic changes that eventually result in the pluripotent state of some infected fibroblasts. Indeed, it has been found that the addition of the epigenetic drug BIX, an inhibitor of the G9a histone methyltransferase, can improve the reprogramming efficiency in neural progenitor cells transduced with Oct3/4-Klf4 to a level comparable to transduction with all 4 factors (Oct3/4, Sox2, Klf4, and c-Myc).

Methods are identical to those described in Example 3, except that human dermal fibroblasts grown as monolayers in DMEM/F12 culture medium supplemented with, non-essential amino acids, L-glutamine and 10% fetal bovine serum are treated with epigenetic drugs (BIX or the demethylation agent 5-azacytidine, among others) for 1 day prior to transfection with recombinant Oct3/4, Sox2, Nanog and Lin-28 and also throughout the iPS induction period prior to selection with hESC medium. Additionally, we may also attempt to affect the epigenetic state of the fibroblast cell by co-transfecting known epigenetic factors such as the recently identified Ronin protein in concert with the reprogramming factors.

In some cases, the recombinant proteins may not be fully functional as expressed in E. coli, due to a lack of post-translational modifications. If this is found to occur, the factors are alternatively expressed using a baculovirus expression system, or the factors may be isolated from nuclear extracts prepared from Human H1 embryonic stem cells using immunoaffinity purification. In this light, we have been successful in growing accutase passaged Human 119 cells to high density on matrigel using Stempro Media (Invitrogen Corp.)

### Example 6

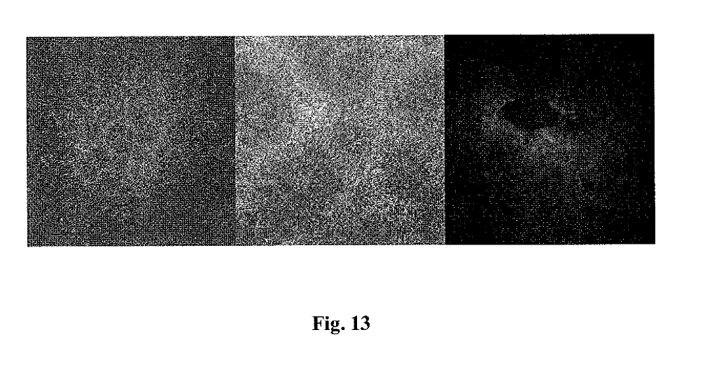

Using the novel protein delivery tool disclosed herein, we show that recombinant human Nanog can be successfully targeted to the nuclei of human fibroblasts. In addition we have also obtained commercially available Sox2 protein (Abnova). As full length Oct3/4 and Lin-28 are not commercially available, we obtained the plasmid constructs for both these proteins and have successfully induced (FIG. 12) and isolated the reprogramming factors.

FIG. 2 illustrates a western blot showing a rapid overwhelming induction of Oct3/4 (Lanes 6, 7 and 8) in the presence of the inducer IPTG, but not in the absence of IPTG (Lanes 2, 3 and 4). Lanes 1 and Lanes 5 represent negative control lanes as aliquots were harvested from both conditions at the time of IPTG addition (T=0 hours). Overnight cultures of E. coli BL21 (DE3) harboring pGEX-4T-1/Oct4 (a generous gift of Dean Tantin, University of Utah), were grown to an OD₆₆₀ of 0.6, and either induced (lanes 5,6,7 and 8) with 1 mm IPTG or Mock-induced (Lanes 1, 2, 3, and 4) at 30 C for time T=0 hours (Lanes 1 and 5) T=6 hours (Lanes 2 and 6), T=18 hrs (Lanes 3 and 7) and T=24 hours (Lanes 4 and 8). 0.1 mL culture aliquots were harvested at the indicate times, recovered by centrifugation, lysed with 4×SDS-PAGE loading dye, boiled and loaded onto a 15% SDS-PAGE Gel. Following SDS Page electrophoresis, proteins were electroblotted to nitrocellulose and Western blotting performed using mouse monoclonal Oct3/4 (C-10) (Santa Cruz Biotechnology, se-5279). Bound antibody was detected using HRP conjugated secondary antibody and ECL.

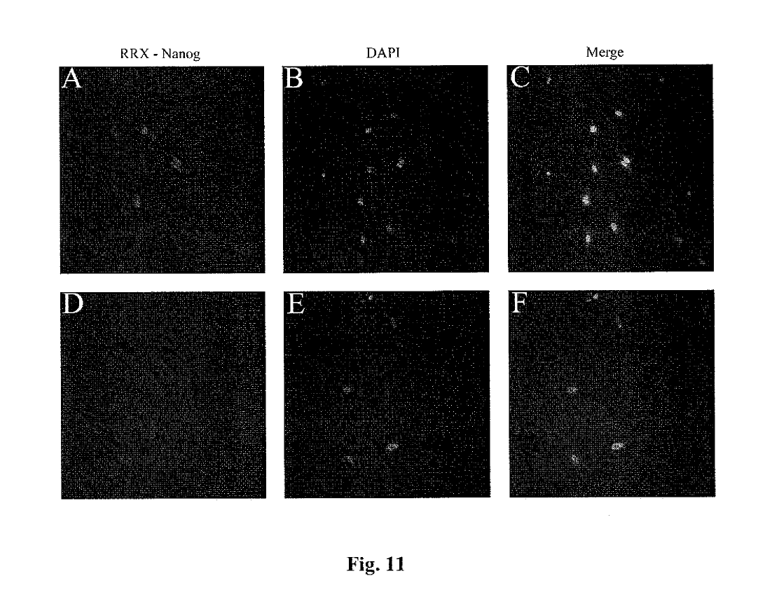

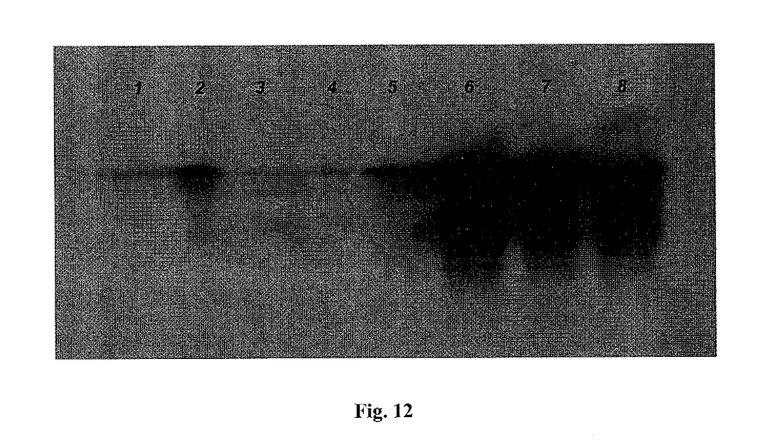

Recombinant full length E. coli produced Nanog (Peprotech) was successfully targeted to the nucleus of human fibroblasts within 5 hours (see FIG. 11) using a novel California-based protein targeting reagent (i.e., Profect P2 reagent). Human fibroblasts (SC-30) in 4-well chamber slides were transfected with Nanog protein in the presence (A, B and C) or absence (D, E and F) of Profect P2 for 1 hour. The Profect P2-Nanog complexes were removed, the cells washed 4 times in serum free media and medium containing 10% serum replaced. 4 hours later, the cells were fixed with 4% paraformaldehyde, blocked in 3% donkey serum and incubated with rabbit polyclonal antibody to human Nanog (H-155; Santa Cruz Biotechnology sc-33759). Bound antibody was detected (panels A and D) with rhodamine red X-conjugated donkey anti-goat Antibody (Jackson Laboratories). Nuclei (panel B and E) were stained using DAPI. Targeting of Nanog to the nucleus is seen in the merged image (Panel C) using the Profect P2 Reagent.

As seen in FIG. 11, Nanog is successfully transfected into the cell in the presence of the Profect P2 reagent (Panel A); however, Nanog is not detectable (Panel D; similar to background staining) in the absence of the Profect P2 reagent. The localization of Nanog is largely nuclear (see merge of Panel A and B; Panel C). No merged signal between DAPI and RRX-Nanog is seen in Panel F as Nanog is not transfected into the cell in the absence of the Profect P2 reagent. This demonstrates that nuclear delivery of the reprogramming factors required for the generation of iPSCs achieved to date using lentiviral transduction may also be achieved using recombinant proteins and a protein targeting reagent such as Profect P2. It is already known that nuclear extracts from one cell type can induce dedifferentiation and reprogramming events in another type. In somatic cell nuclear transfer (SCNT), preexisting reprogramming factors in the egg cytoplasm convert the epigenome of a somatic cell into that of an embryonic cell. Therefore delivery of the sets of reprogramming factors identified in the original pioneering description of lentiviral derived iPSCs are also likely sufficient to induce reprogramming independent of lentiviral infection.

### Example 7

### Differentiation of Human Pluripotent Stem Cells into Neural Stem Cells (NSCs)

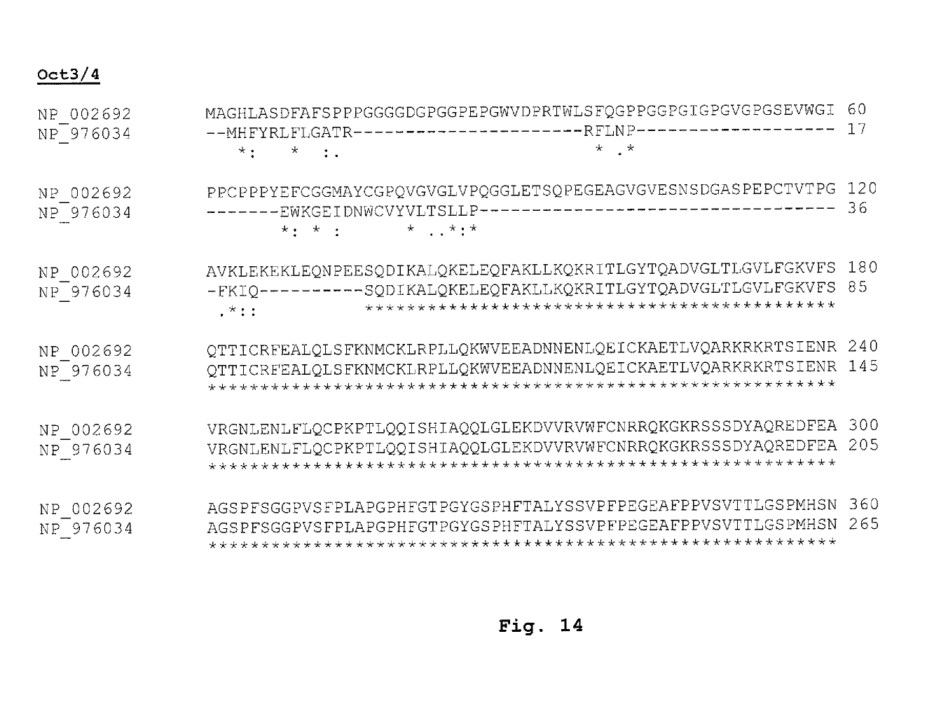

Methods for differentiation of hESCs down the neural lineage are detailed in a recent neural differentiation methods review article (Schwartz, P. H. et al. 2008 Methods 45:142-158). Referring to FIG. 13, neurally-induced embryoid bodies derived from ePSCs and plated onto Matrigel show classic neural rosette formation, which stain positively for the NSC markers Sox1 and N-cadherin.

It is understood that the examples and embodiments described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

What is claimed is:

1. A method of de-differentiating somatic cells to an embryonic stem cell state comprising direct delivery of a protein into said somatic cell, wherein the protein effects de-differentiation of said somatic cell to an embryonic stem cell phenotype.

2. The method of claim 1, wherein said protein is a gene product of a gene listed in Table 1.

3. The method of claim 2 wherein said protein is selected from the group consisting of Oct3/4, Sox2, Nanog, Stat3, E-Ras, c-Myc, Klf4, β-catenin and Lin28.

4. The method of claim 1 wherein said protein is a mutant, variant or a derivative of a protein or polypeptide that is able to induce and maintain the embryonic stem cell phenotype.

5. The method of claim 1, wherein said protein is selected from the group consisting of SEQ ID NO: 1, 2, 3, 4, 5, 6, 7, 8, 9, 10, 11 and 12, or wherein said protein has at least 95% sequence identity to SEQ ID No: 1, 2, 3, 4, 5, 6, 7, 8, 9, 10, 11 or 12.

6. The method of claim 1 wherein said proteins are delivered to said cells with a Profect protein delivery reagent selected from the group consisting of Profect-P1 and Profect-P2.

7. The method of claim 1 wherein the protein is delivered to a cell in cell culture.

8. The method of claim 1 wherein said cell is a mammalian cell.

9. The method of claim 7 wherein said cell is a fibroblast cell.

10. The method of claim 7 wherein said mammalian cell is human.

11. The method of claim 1 wherein the protein that effects de-differentiation of the somatic cell to an embryonic stem cell phenotype is identified by differential gene expression analysis.


SEQUENCE LISTING (14 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 4 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 5 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 6 - protein, 769 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGAAPYLKTKFICVTPTTCSNTIDLPMSPRTLDSLMQFGNNGEGAEPSAGGQFESLTFDMELTSECATSPM

SEQ ID NO: 7 - protein, 722 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPFIDAVWK

SEQ ID NO: 8 - protein, 233 aa, Homo sapiens
MELPTKPGTFDLGLATWSPSFQGETHRAQARRRDVGRQLPEYKAVVVGASGVGKSALTIQLNHQCFVEDHDPTIQDSYWKELTLDSGDCILNVLDTAGQAIHRALRDQCLAVCDGVLGVFALDDPSSLIQLQQIWATWGPHPAQPLVLVGNKCDLVTTAGDAHAAAAALAHSWGAHFVETSAKTRQGVEEAFSLLVHEIQRVQEAMAKEPMARSCREKTRHQKATCHCGCSVA

SEQ ID NO: 9 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 10 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 11 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 12 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 13 - protein, 227 aa, Mus musculus
MALPTKSSILDLSSGTPCTRSPEESHEAWAQCKDAGRQLPEYKAVVVGASGVGKSALTIQMTHQCFVKDHDPTIQDSYWKEVARDNGGYILNVLDTSGQDIHRALRDQCLASGDGVLGVFALDDPSSLDQLQQIWSTWTPHHKQPLVLVGNKCDLVTTAGDAHAAAALLAHKLGAPLVKTSAKTRQGVEEAFALLVHEIQRAQEAVAESSKKTRHQKAVCSCGCSVA

SEQ ID NO: 14 - protein, 189 aa, Rattus norvegicus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS